(19) United States
(12) Patent Application Publication
(10) Pub. No.: US 2006/0019893 A1
(43) Pub. Date: Jan. 26, 2006

(54) Factor VIIa variants

(75) Inventors: Henry R. Maun, San Francisco, CA (US); Charles Eigenbrot, Burlingame, CA (US); Robert A. Lazarus, Millbrae, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/159,647

(22) Filed: Jun. 23, 2005

Related U.S. Application Data
(60) Provisional application No. 60/585,499, filed on Jul. 2, 2004.

(51) Int. Cl. C07K 14/755

(52) U.S. Cl. 514/12; 530/383

(57) ABSTRACT

Novel compounds are provided which modulate a FVIIa mediated or associated process or event such as the catalytic conversion of FX to FXa, FVII to FVIIa or FIX to FIXa. In particular aspects, the compounds of the invention are variants of Factor VIIa (FVIIa). Pharmaceutical compositions are also provided which comprise the novel compounds as well as their use in diagnostic, therapeutic, and prophylactic methods.

### RELATED APPLICATION

This application claims priority to and the benefit of U.S. Provisional Application Ser. No. 60/585,499, filed Jul. 2, 2004, the specification of which is incorporated herein in its entirety.

### FIELD OF THE INVENTION

This invention relates to novel compositions comprising amino acid sequence variants of Factor VIIa. The Factor VIIa variants can modulate procoagulation activity in the presence or absence of tissue factor. The invention also relates to pharmaceutical compositions comprising the novel compositions as well as their use in diagnostic, therapeutic, and prophylactic methods.

### BACKGROUND OF THE INVENTION

Coagulation is the biological process of blood clot formation involving many different serine proteases as well as their essential cofactors and inhibitors. See, e.g., Davie, E. W., et al., “The coagulation cascade: Initiation, maintenance, and regulation” Biochemistry 30:10363-10370 (1991); Nemerson, Y. “Tissue factor and hemostasis” Blood 71:1-8 (1988); Broze Jr., G. J. “Tissue factor pathway inhibitor and the revised hypothesis of blood coagulation” Trends Cardiovasc. Med. 2:72-77 (1992); Rapaport, S. I. and Rao, L. V. M. “The tissue factor pathway: How it has become a ‘Prima Ballerina’” Thromb. Haemost. 74:7-17 (1995); Davie, E. W. “Biochemical and molecular aspects of the coagulation cascade.” Thromb. Haemost. 74:1-6 (1995); Giesen, P. L. A., et al., “Blood-borne tissue factor: Another view of thrombosis.” Proc. Natl. Acad. Sci. U.S.A. 96:2311-2315 (1999); and, Mann, K. G. “Thrombin formation” Chest 124:4S-10S (2003). It is initiated by exposure of Factor VII (FVII) and Factor VIIa (FVIIa) to its membrane bound cofactor, tissue factor (TF), resulting in production of Factor FXa (FXa) and more FVIIa. The process is propagated upon production of Factor IXa (FIXa) and more FXa that, upon binding with their respective cofactors FVIIIa and FVa, form platelet bound complexes, ultimately resulting in the formation of thrombin and a fibrin clot. Thrombin also serves to further amplify coagulation by activation of cofactors such as FV and FVII and zymogens such as Factor XI. Moreover, thrombin activates platelets leading to platelet aggregation, which is necessary for the formation of a hemostatic plug.

Factor VIIa (FVIIa) is a two-chain, 50 kilodalton (kDa), vitamin K-dependent, plasma serine protease. Factor VIIa is generated by proteolysis of a single peptide bond from its single chain zymogen, Factor VII, which is present at approximately 0.5 μg/ml in plasma. The conversion of zymogen Factor VII into the activated two-chain molecule occurs by cleavage of an internal peptide bond. In human Factor VII, the cleavage site is at Arg152-Ile153 (Hagen et al., Proc. Natl. Acad. Sci. USA 83:2412-2416 (1986); and, Thim et al., Biochem. 27:7785-7793 (1988)). In the presence of calcium ions, Factor VIIa binds with high affinity to TF.

TF is a 263 amino acid residue glycoprotein composed of a 219 residue extracellular domain, a single transmembrane domain, and a short cytoplasmic domain (Morrissey, J. H., et al., Cell 50:129-135 (1987)). The TF extracellular domain is composed of two fibronectin type III domains of about 105 amino acids each. The binding of FVIIa is mediated entirely by the TF extracellular domain (Muller et al., Biochem. 33:10864-10870 (1994); Gibbs et al., Biochem. 33:14003-14010 (1994); Ruf et al., Biochem. 33:1565-1572 (1994)). The structure of the TF extracellular domain has been determined by x-ray crystallography (Harlos et al., Nature 370:662-666 (1994); Muller et al., Biochemistry 33:10864 (1994)). The TF extracellular domain has also has been extensively characterized by alanine scanning mutagenesis (Kelley et al., Biochemistry, 34:10383-10392 (1995); Gibbs et al., (1994) supra; Ruf et al., (1994) supra). Residues in the area of amino acids 16-26 and 129-147 contribute to the binding of FVIIa as well as the coagulant function of the molecule. Residues Lys20, Trp45, Asp58, Tyr94, and Phe140 make a large contribution (1 kcal/mol) to the free energy (AG) of binding to FVIIa (Kelley et al., (1995) supra).

TF is expressed constitutively on cells separated from plasma by the vascular endothelium (Carson, S. D. and J. P. Brozna, Blood Coag. Fibrinol. 4:281-292 (1993)). Its expression on endothelial cells and monocytes is induced by exposure to inflammatory cytokines or bacterial lipopolysaccharides (Drake et al., J. Cell Biol. 109:389 (1989)). Upon tissue injury, the exposed extracellular domain of TF forms a high affinity, calcium dependent complex with FVII. Once bound to TF, FVII can be activated by peptide bond cleavage to yield serine protease FVIIa. The enzyme that catalyzes this step in vivo has not been elucidated, but in vitro FXa, thrombin, TF•FVIIa and FIXa can catalyze this cleavage (Davie, et al., (1991) supra). FVIIa has only weak activity upon its physiological substrates FX and FIX whereas the TF•FVIIa complex rapidly activates FX and FIX.

The TF•FVIIa complex constitutes the primary initiator of the extrinsic pathway of blood coagulation (Carson, S. D. and Brozna, J. P., Blood Coag. Fibrinol. 4:281-292 (1993); Davie, E. W. et al., (1991) supra; Rapaport, S. I. and L. V. M. Rao, Arterioscler. Thromb. 12:1111-1121 (1992)). The complex initiates the extrinsic pathway by activation of FX to Factor Xa (FXa), FIX to Factor IXa (FIXa), and additional FVII to FVIIa. The action of TF•FVIIa leads ultimately to the conversion of prothrombin to thrombin, which carries out many biological functions (Badimon, L. et al., Trends Cardiovasc. Med. 1:261-267 (1991)). Among the most important functions of thrombin is the conversion of fibrinogen to fibrin, which polymerizes to form a clot. The TF•FVIIa complex also participates as a secondary factor in extending the physiological effects of the contact activation system.

The initiation and subsequent regulation of coagulation is complex, since maintenance of hemostasis is crucial for survival. See, e.g., Mann, K. G. (2003) supra; and. Lawson, J. H. and Murphy, M. P. “Challenges for providing effective hemostasis in surgery and trauma” Semin. Hematol. 41 (suppl. 1):55-64 (2004). There is an exquisite balance between hemostasis (normal clot formation and dissolution) and thrombosis (pathogenic clot formation). Serious clinical conditions involving aberrations in coagulation include deep vein thrombosis, myocardial infarction, pulmonary embolism, stroke and disseminated intravascular coagulation (in sepsis). There are also many bleeding coagulopathies where there is insufficient clot formation. These include hemophilia A (FVIII deficiency) or hemophilia B (FIX deficiency), where procoagulant therapy is required. The challenge in this therapeutic area is to operate in the narrow window between too much and too little coagulation.

The use of exogenous FVIIa as a therapeutic agent has been shown to induce hemostatsis in patients with hemophilia A and B. See, e.g., Hedner, U. (2001) “Recombinant factor VIIa (Novoseven®) as a hemostatic agent” Semin. Hematol. 38 (suppl. 12):43-47 (2001); and, Hedner, U. “Dosing with recombinant factor VIIa based on current evidence” Semin. Hematol. 41 (Suppl. 1):35-39 (2004). It also has been used to treat bleeding in patients with liver disease, anticoagulation-induced bleeding, surgery, thrombocytopenia, thrombasthenia, Bemard-Soulier syndrome, von Willebrand disease, and other bleeding disorders. See, e.g., Midathada, M. V., et al., (2004) “Recombinant factor VIIa in the treatment of bleeding” Am. J. Clin. Pathol. 121:124-137 (2004). The precise mechanism of action of exogenously added FVIIa is a matter of some debate as to whether TF is required because evidence for both TF-dependent and TF-independent FVIIa clotting activity has been presented. See, e.g., Lisman, T. and de Groot, P. G. (2003) “Mechanism of action of recombinant factor VIIa” J. Thromb. Haemost. 1:1138-1139 (2003); Butenas, S., et al., “How factor VIIa works in hemophilia” J. Thromb. Haemost. 1, 1158-1160 (2003); Butenas, S., et al., “Influence of factor VIIa and phospholipids on coagulation in “acquired” hemophilia” Arterioscler. Thromb. Vasc. Biol. 23:123-129 (2003); Monroe, D. M. and Roberts, H. R. (2003) “Mechanism of action of high-dose factor VIIa: points of agreement and disagreement” Arterioscler. Thromb. Vasc. Biol. 23:8-9 (2003); and, Butenas, S. and Mann, K. G. “Response: Mechanism of action of high-dose factor VIIa” Arterioscler. Thromb. Vasc. Biol. 23:10 (2003). Treatment with recombinant FVIIa typically requires relatively high plasma concentrations. Thus, the need of novel variants of FVIIa with enhanced enzymatic activities and/or altered properties, e.g., enzymatic activity in the absence of TF, compared to native or recombinant FVIIa are of interest. The invention addresses these and other needs, as will be apparent upon review of the following disclosure.

### SUMMARY OF THE INVENTION

The invention provides compositions comprising sequence, e.g., nucleic acid and amino acid, variants of FVIIa. The FVIIa variants have enzymatic activity either in the presence or absence of TF. The invention provides compounds and compositions which induce a FVII/FVIIa mediated or associated process such as the catalytic conversion of FVII to FVIIa, FIX to FIXa, or FX to FXa and thereby initiating initial events of the extrinsic pathway of blood coagulation. In addition, the compositions of the invention are capable of inducing procoagulation. The compositions of the invention are therefore useful in therapeutic and prophylactic methods for inducing FVIIa mediated or associated processes.

According to certain aspects of the invention, a FVIIa variant is provided having an amino acid sequence derived from a mammalian FVIIa protein (e.g., a human FVIIa protein), where at least two non-cysteine amino acid residues are substituted with a cysteine amino acid. In certain aspects of the invention, a Factor VIIa (FVIIa) variant comprises an amino acid sequence derived from a mammalian FVIIa protein, where at least two amino acid residues are substituted with an amino acid (e.g., a cysteine amino acid, an unnatural amino acid or modified amino acid) that locks A2-strand of FVIIa to B2-strand of FVIIa. In one embodiment, the at least two amino acid residues form a disulfide bond. In certain embodiments, the two amino acid residues correspond to a human amino acid residue pair, e.g., S136 and V160, L137 and N159, V138 and V160, S139 and V157, F135 and N159, F135 and P161, V138 and L158, F135 and M156, and/or, V138 and L155. The chymotrypsinogen residue numbering convention is used. In a further embodiment, multiple amino acid residue pairs are substituted, e.g., two or more pairs, or three or more pairs, or four or more pairs, or five or more pairs, etc., to lock the A2-strand of FVIIa to B2-strand of FVIIa. In certain aspects of the invention, the FVIIa variant comprises an enhanced activity in the absence of tissue factor protein compared to a naturally occurring mammalian FVIIa protein or recombinant non-variant FVIIa protein.

In one embodiment, the invention provides a Factor Vila (FVIIa) variant comprising an amino acid sequence derived from a mammalian FVIIa protein, wherein at least two amino acid residues are substituted with a cysteine amino acid, which correspond to a human amino acid residue pair S136 and V160. In one embodiment, a Factor VIIa (FVIIa) variant is provided that comprises an amino acid sequence derived from a mammalian FVIIa protein, where at least two amino acid residues are substituted with a cysteine amino acid, which correspond to a human amino acid residue pair L137 and N159. In one embodiment, a Factor VIIa (FVIIa) variant of the invention comprises an amino acid sequence derived from a mammalian FVIIa protein, where at least two amino acid residues are substituted with a cysteine amino acid, which correspond to a human amino acid residue pair V138 and V160. In one embodiment, a Factor VIIa (FVIIa) variant includes an amino acid sequence derived from a mammalian FVIIa protein, where at least two amino acid residues are substituted with a cysteine amino acid, which correspond to a human amino acid residue pair S139 and V157.

The invention additionally provides for FVIIa variants having further amino acid substitutions. In further embodiment, a FVIIa variant of the invention includes at least one additional, optionally, two or more, optionally, three or more, optionally, four or more, etc., amino acid substitutions. In certain embodiments, the additional amino acid substitution(s) contributes to FVIIa variant activity. In certain aspects of the invention, the additional amino acid substitution corresponds to a change in the human amino acid residue, e.g., (Chymotrypsinogen numbering is used; (FVIIa continuous numbering scheme is in italics in parenthesis)); E17 (E154), V21 (V158), F135 (F278), S136 (S279), L137 (L280), V138 (V281), S139 (S282), E154 (E296), L155 (L297), M156 (M298), V157 (V299), L158 (L300), N159 (N301), V160 (V302), L163 (L305), M164 (M306), D167 (D309), S170b (S314), K188 (K337) and/or F225 (F374). For example, the change in the human amino acid residue includes, e.g., V21D (V158D), V21E (V158E), V21N (V158N), E154V (E296V), E1541 (E296I), E154R (E296R), M156Q (M298Q), M156K (M298K), L163V (L305V), M164D (M306D), D167S (D309S), S170bE (S314E), K188A (K337A), and/or F225Y (F374Y). Other mutations in the 99 loop and 170 loop can also be present in FVIIa variants of the invention. Modifications in the Gla domain of FVIIa, e.g., to obtain higher membrane binding affinity and FVIIa activity, can also be present.

In one embodiment, the compositions of the invention are polypeptides. The invention also encompasses a composition comprising an isolated nucleic acid, preferably DNA, encoding a polypeptide of the invention. In certain aspects of the invention, the invention further comprises an expression control sequence operably linked to the DNA molecule. In one embodiment, an expression vector, e.g., a plasmid, comprises the DNA molecule, where the control sequence is recognized by a host cell. Vectors and host cells with the introduced vector are also provided in the invention. Methods of producing a FVIIa variant are also included in the invention. For example, a method includes culturing the host cell with the DNA encoding a FVIIa variant of the invention, under conditions suitable for expression of the FVIIa variant, thereby producing the FVIIa variant. In one aspect of the invention, the method further comprises recovering the FVIIa variant from the culture medium.

The invention further includes therapeutic applications for the compositions described herein. In certain embodiments of the invention, the invention-includes a pharmaceutical composition comprising a pharmaceutically acceptable excipient and a FVIIa variant of the invention. Pharmaceutical compositions comprising these molecules can be used in the treatment or prophylaxis of thrombotic or coagulopathic related diseases or disorders including hereditary deficiencies in coagulation factors, vascular disease, and inflammatory responses. See also, the definition of disorders, described herein. The applications include, e.g., methods of altering procoagulation (e.g., the induction of procoagulation) in a mammal (e.g., human) comprising administering an effective amount of a pharmaceutical composition of the invention to the mammal. Additional agents for bleeding disorders can also be administered in combination with the FVIIa variants of the invention.

### BRIEF DESCRIPTION OF THE FIGURES

FIG. 1, Panels A and B schematically illustrate a FVIIa variant disulfide lock strategy. In Panel A, registration of strands A2 and B2 in FVII and FVIIa are shown. Locking amino acids, e.g., cysteine pairs, can be introduced at the residues that can form a pair, e.g., disulfide, and lock the registration of the strand in the active or inactive state. In Panel B, the registration of strands A2 and B2 in the TF•FVIIa-like active enzyme state and the zymogen FVII-like state are illustrated. The link between cysteine pairs is depicted in bold lines that are introduced at residue pairs that could form a disulfide in the strand registration of the TF•FVIIa-like active state. The distance between C_{α} atoms in Å for the TF•FVIIa-like registration and (zymogen FVII) registrations are noted in the table with arrows pointing towards the engineered disulfide residue pair. In Panels A and B, hydrogen bonds are shown with dashed lines between residues in the A2 and B2 strands. The Leu-X-Val-Leu-X-Val residues important for reregistration in the zymogen and TF•FVIIa-like conformations are depicted in bold ovals for both registrations.

FIG. 2 illustrates kinetics of FVIIa variants or wild type FVIIa with the peptide substrate S2765 to measure amidolytic activity. Individual kinetic analysis for amidolytic activity of S-2765 with 30 nM wildtype FVIIa (●) and 30 nM FVIIa variants (all normalized by active site titration) 136:160 (▴), 137:159 (♦), 138:160 (▾), and 139:157 (▪) is illustrated.

FIG. 3 illustrates relative TF-dependent clotting of FVIIa variants or wild type in FVII deficient plasma. Relative clotting times are normalized to the clotting time in FVII deficient plasma. Data is shown for wildtype FVIIa (●) and FVIIa variants 136:160 (▴), 137:159 (♦), 138:160 (▾), and 139:157 (▪).

FIG. 4 illustrates an example of SDS-PAGE gel analysis of a FVIIa mutant. Purified FVII mutant 139:157 was run on SDS-PAGE gels under both (A) nonreduced and (B) reduced conditions. FVIIa mutant 139:157 activated by FXa as described herein was run on a (C) reduced SDS-PAGE gel where heavy and light chains are indicated. Molecular mass markers are shown in kDa.

FIG. 5 illustrates amidolytic activity of FVII variants. The fold increases in amidolytic activity (Vₘₐₓ/Kₘ) for FVIIa disulfide locked variants in the absence of sTF relative to wildtype are shown. Data for variants with S-2765 is shown in white boxes and with Spectrozyme fXa in gray boxes; the fold increase is shown above their respective columns.

FIG. 6, Panels A and B illustrate trypsin digestion of FVIIa, which produces a Gla domain peptide (tryptic peptide: ANAFLXLRPGSLXRXCKXXQCSFXXARXIFK where X=Gla) containing nine of the 10 sites of potential Gla modification. Representative MALDI-TOF analysis shows similar patterns and extents of modifications between the wild-type (Panel A) and S139C:V157C mutant (Panel B).

### DETAILED DESCRIPTION

Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

Unless defined otherwise, all scientific and technical terms are understood to have the same meaning as commonly used in the art to which they pertain. For the purpose of the invention, the following terms are defined below.

Abbreviations used throughout the description include: TF for tissue factor; —FVIIa for Factor VIIa; TF•FVIIa for tissue factor•Factor VIIa complex; FVII for zymogen factor VII; FIX for Factor IX; FIXa for Factor IXa; FX for Factor X; FXa for Factor Xa; FXIa for Factor XIa; LMWH for low-molecular weight heparin; Gla for γ-carboxyglutamic acid; EGF for epidermal growth factor; sTF for soluble tissue factor comprising the extracellular domain, residues 1-219; rlTF for relipidated recombinant human tissue factor residues 1-243; MS for mass spectrometry; SDS-PAGE for sodium dodecyl sulfate—polyacrylamide gel electrophoresis.

The term “FVIIa” refers to FVIIa protein, along with naturally occurring allelic and processed forms thereof. The amino acid positions in the FVIIa are numbered based on chymotrypsinogen, e.g., to ease comparisons among homologous proteins. The residue position using the continuous FVIIa numbering scheme is found in italics. A conversion of chymotrypsin numbering to FVII protein is known in the art and is found in Table 1. The terms “wild type FVII” and “wild type FVIIa” are used to refer to a polypeptide having an amino acid sequence corresponding to a naturally occurring mammalian FVII or FVIIa or a recombinant FVII or FVIIa having an amino acid sequence of a naturally occurring FVII or FVIIa which is capable of inducing blood coagulation. Naturally occurring FVII or FVIIa includes human species as well as other animal species such as rabbit, rat, porcine, non human primate, equine, murine, bovine, and ovine FVII or FVIIa. The amino acid sequences of the mammalian FVII or FVIIa proteins are generally known or obtainable through conventional techniques.

The term “FVIIa variant” as used herein refers to an FVIIa polypeptide which includes at least two or more amino acid substitutions in the native FVIIa sequence. In certain embodiments of the invention, the amino acid substitutions can lock the FVIIa variant in a desired conformation. Typically, the at least two amino acid substitutions are cysteine residues, which then can form a disulfide bond to lock the FVIIa configuration. Other residues can also be used, e.g., modified amino acids, unnatural amino acids, etc. The residue position number can be used in conjunction with the single letter nomenclature to designate the residue at which a substitution is made in the FVIIa variants of the invention. For example, in a FVIIa variant in which glutamine (Q) replaces methionine (M) at residue position number 156 of the naturally occurring human FVIIa numbered according to chymotrypsin numbering, the nomenclature “M156Q” or the like is used.

TABLE 1
FVIIa numbering corresponding to Chymotrypsinogen Numbering

| | FVIIa continuous | chymotrypsinogen |
|---|---|---|
| | numbering | numbering |
| A2 Strand | | |
| | F278 | F135 |
| | S279 | S136 |
| | L280 | L137 |
| | V281 | V138 |
| | S282 | S139 |
| | G283 | G140 |
| B2 Strand | | |
| | L295 | L153 |
| | E296 | E154 |
| | L297 | L155 |
| | M298 | M156 |
| | V299 | V157 |
| | L300 | L158 |
| | N301 | N159 |
| | V302 | V160 |
| | P303 | P161 |
| Arginines for A2 and B2 | | |
| strand tryptic digest | | |
| analysis | | |
| | R277 | R134 |
| | R290 | R147 |
| | R304 | R162 |

The term “amino acid” within the scope of the invention is used in its broadest sense and is meant to include the naturally occurring L α-amino acids or residues, and unnatural or modified amino acids. The commonly used one- and three-letter abbreviations for naturally occurring amino acids are used herein (Lehninger, A. L., Biochemistry, 2d ed., pp. 71-92, (Worth Publishers, New York, N.Y., 1975). The term includes D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio, The Peptides: Analysis, Synthesis, Biology, Gross and Meiehofer, Eds., Vol. 5, p. 341 (Academic Press, Inc., New York, N.Y., 1983), which is incorporated herein by reference.

“Naturally occurring amino acid residues” (i.e. amino acid residues encoded by the genetic code) may be selected from the group consisting of: alanine (Ala) (A); arginine (Arg) (R); asparagine (Asn)(N); aspartic acid (Asp) (D); cysteine (Cys) (C); glutamine (Gln) (O); glutamic acid (Glu) (E); glycine (Gly) (G); histidine (His) (H); isoleucine (Ile) (I): leucine (Leu) (L); lysine (Lys) (K); methionine (Met) (M); phenylalanine (Phe) (F); proline (Pro) (P); serine (Ser) (S); threonine (Thr) (T); tryptophan (Trp) (W); tyrosine (Tyr) (Y); and valine (Val) (V). A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include, e.g., norleucine, ornithine, norvaline, homoserine and other amino acid residue analogues such as those described in Ellman et al. Meth. Enzym. 202:301-336 (1991) & U.S. Patent applications 20030108885 and 20030082575. Briefly, these procedures involve activating a suppressor tRNA with a non-naturally occurring amino acid residue followed by in vitro or in vivo transcription and translation of the RNA. See, e.g., U.S. Patent applications 20030108885 and 20030082575; Noren et al. Science 244:182 (1989); and, Ellman et al., supra.

Additional FVIIa variants are those in which at least one additional amino acid residue in the FVIIa variant of the invention has been removed and a different residue inserted in its place. Such substitutions may be made in accordance with those shown in Table 2, and described herein. FVIIa variants can also comprise unnatural amino acids as described herein.

The term “conservative” amino acid substitution as used within this invention is meant to refer to amino acid substitutions which substitute functionally equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide.

Amino acids may be grouped according to similarities in the properties of their side chains (in A. L. Lehninger, in Biochemistry, second ed., pp. 73-75, Worth Publishers, New York (1975)):
(1) non-polar: Ala (A), Val (V), Leu (L), ile (I), Pro (P), Phe (F), Trp (W), Met (M)
(2) uncharged polar: Gly (G), Ser (S), Thr (T), Cys (C), Tyr (Y), Asn (N), Gin (O)
(3) acidic: Asp (D), Glu (E)
(4) basic: Lys (K), Arg (R), His(H)

Alternatively, naturally occurring residues may be divided into groups based on common side-chain properties:
(1) hydrophobic: Norleucine, Met, Ala, Val, Leu, Ile;
(2) neutral hydrophilic: Cys, Ser, Thr, Asn, Gln
(3) acidic: Asp, Glu;
(4) basic: His, Lys, Arg;
(5) residues that influence chain orientation: Gly, Pro; and,

(6) aromatic: Trp, Tyr, Phe.

TABLE 2

| Original | Exemplary | Typical |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

By “substitution” of any amino acid is meant that an amino acid of the wild-type FVIIa has been replaced or modified by chemical or enzymatic or other appropriate means with a moiety other than a wild-type amino acid.

The term “activity” is used to refer to a FVII/FVIIa mediated or associated activity or event, or TF•FVIIa mediated or associated process or event, which is any event which requires the presence of FVIIa.

The terms “tissue factor protein” and “wild type tissue factor” are used to refer to a polypeptide having an amino acid sequence corresponding to a naturally occurring mammalian tissue factor or a recombinant tissue factor having an amino acid sequence of a naturally occurring tissue factor which is capable of inducing blood coagulation through its interaction with plasma FVII/FVIIa. Naturally occurring TF includes human species as well as other animal species such as rabbit, rat, porcine, non human primate, equine, murine, bovine, and ovine tissue factor. The amino acid sequences of the mammalian tissue factor proteins are generally known or obtainable through conventional techniques. The human sequence as well as the number given to the amino acids are those described by Morrissey, J. H., et al., Cell 50:129-135 (1987). Synthetic and recombinant tissue factor proteins are generally known in the art and included, for example, sTF (Waxman et al., (1992) Biochemistry 31: 3998-4005 (1992); Neuenschwander, P. F. and Morrissey, J. H. J. Biol. Chem. 267:14477-14482 (1992)).

A “disorder” is any condition that would benefit from treatment with the FVIIa variant of the invention. This includes chronic and acute disorders or diseases including those pathological conditions which predispose the mammal to the disorder in question. Non-limiting examples of disorders to be treated herein include, but are not limited to, e.g., thrombotic or coagulopathic related diseases or disorders, Hemophilia A (FVII deficiency), Hemophilia B (factor IX deficiency), Hemophilia C (factor XI deficiency), hemophilia with inhibitors and acquired inhibitors of factors VIII and X, Christmas disease (Factor IX deficiency), Stuart factor disease (factor X deficiency), SPCA (serum prothrombin conversion accelerator) deficiency (factor VII deficiency), clotting disorders due to Vitamin K deficiencies, liver disease, liver transplantation, renal failure, intractable bleeding, fibrinogen deficiencies (liver disease, disseminated intravascular coagulation (DIC), L-asparaginase therapy, rattlesnake bites), clotting factor deficiencies, circulating anticoagulants (e.g., in the case of lymphoma, SLE, idiopathic), massive transfusion (e.g., dilutional coagulopathy), anticoagulation-induced bleeding, surgery, platelet disorders, thrombocytopenia, thrombasthenia, von Willebrand disease, Bernard-Soulier syndrome, vascular disease, inflammatory responses, bone marrow problems, bone marrow transplantation, pregnancy bleeding disorders, traumatic bleeding, and other bleeding disorders, etc.

As used herein, the term “parenteral” refers to introduction of a compound of the invention into the body by other than the intestines, and in particular, intravenous (i.v.), intraarterial (i.a.), intraperitoneal (i.p.), intramuscular (i.m.), intraventricular, and subcutaneous (s.c.) routes.

The term “treatment” as used within the context of the invention is meant to include therapeutic treatment as well as prophylactic, or suppressive measures for the disease or disorder. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease. Further, administration of the agent after onset and after clinical symptoms has developed where administration affects clinical parameters of the disease or disorder, such as the degree of tissue injury or the amount or extent of leukocyte trafficking and perhaps amelioration of the disease, comprises “treatment” of the disease.

Administration “in combination with” one or more further therapeutic agents includes simultaneous (concurrent) and consecutive administration in any order.

“Mammal” for purposes of treatment refers to any animal classified as a mammal, including humans, domestic and farm animals, and zoo, sports, or pet animals, such as dogs, horses, cats, cows, sheep, pigs, etc. Typically, the mammal is a human. Included in the definition are mammals already having the disease or disorder, including those in which the disease or disorder is to be prevented.

The term “effective amount” or “therapeutically effective amount” refers to an amount of a drug effective to treat a disease or disorder in a mammal.

FVIIa Variants

Critical conformational changes occur upon proteolytic processing of zymogen FVII to Factor VIIa (FVIIa), a serine protease involved in the initiation of the coagulation cascade. However, maximal enzymatic activity of FVIIa towards its biological substrates requires further conformational changes induced upon binding to tissue factor (TF). One of the key conformational changes affecting the zymogenicity of FVIIa involves a unique three-residue shift causing reregistration of beta strands A2 and B2 in the zymogen and protease forms. By locking the conformation of these strands into a FVIIa-like or zymogen-like state, a FVIIa variant can be produced by, e.g., introducing cysteine residue pairs at the appropriate positions to form a new disulfide bond. In certain embodiments, the FVIIa variants of the invention have an enhanced enzymatic activity compared to FVIIa alone or TF•FVIIa.

Approaches to enhance the enzymatic activity of FVIIa stem from the observation that FVIIa has key allosteric linked regions involving the TF binding site, the active site and the macromolecular binding site. See, e.g., Ruf, W. and Dickinson, C. D. “Allosteric regulation of the cofactor-dependent serine protease coagulation factor VIIa” Trends Cardiovasc. Med. 8:350-356 (1998). The structures of TF•FVIIa complex (see, e.g., Banner, D. W., et al., (1996) “The crystal structure of the complex of blood coagulation factor Vila with soluble tissue factor” Nature 380:4146 (1996); and, Zhang, E., et al., “Structure of extracellular tissue factor complexed with Factor VIIa inhibited with a BPTI mutant” J. Mol. Biol. 285:2089-2104 (1999)), FVIIa (see, e.g., Dennis, M. S., et al., “Peptide exosite inhibitors of factor VIIa as anticoagulants” Nature 404:465-470 (2000); Kemball-Cook, G., et al., “Crystal structure of active site-inhibited human coagulation factor VIIa (des-Gla)”. J. Struct. Biol. 127: 213-223 (1999); Pike, A. C. W., et al., “Structure of human factor VIIa and its implications for the triggering of blood coagulation” Proc. Natl. Acad. Sci. USA 96:8925-8930 (1999); and, Sichler, K., et al., “Crystal structures of uninhibited factor VIIa link its cofactor and substrate-assisted activation to specific interactions” J. Mol. Biol. 322:591-603 (2002)) and the zymogen FVII (see, e.g., Eigenbrot, C., et al., “The factor VII zymogen structure reveals reregistration of β-strands during activation” Structure 9:627-636 (2001)) have provided a structural basis to begin to understand this allostery. Comparison of the structural differences between TF•FVIIa, FVIIa and FVII have led to several recent advances. These have focused on the roles of allostery and zymogenicity of FVIIa in different states. See, e.g., Petrovan, R. J. and Ruf, W. “Residue Met156 contributes to the labile enzyme conformation of coagulation factor VIIa” J. Biol. Chem. 276:6616-6620 (2001); Persson, E., et al., “Substitution of aspartic acid for methionine-306 in factor VIIa abolishes the allosteric linkage between the active site and the binding interface with tissue factor” Biochemistry 40:3251-3256 (2001); Petrovan, R. J. and Ruf, W. “Role of zymogenicity-determining residues of coagulation factor VI/VIIa in cofactor interaction and macromolecular substrate recognition” Biochemistry 41:9302-9309 (2002); and, Toso, R., et al., “Factor VII mutant V154G models a zymogen-like form of factor VIIa” Biochem. J. 369, 563-571 (2003). Further understanding has led to engineered variants with improved catalytic activity. See, e.g., Persson, E., et al. “Substitution of valine for leucine 305 in factor VIIa increases the intrinsic enzymatic activity” J. Biol. Chem. 276:29195-29199 (2001); Persson, E., et al., (2001) “Rational design of coagulation factor VIIa variants with substantially increased intrinsic activity” Proc. Natl. Acad. Sci. U.S.A. 98:13583-13588 (2001); Soejima, K., et al., “The 99 and 170 loop-modified factor VIIa mutants show enhanced catalytic activity without tissue factor” J. Biol. Chem. 277:49027-49035 (2002); Persson, E. and Olsen, 0. H. “Assignment of molecular properties of a superactive coagulation factor VIIa variant to individual amino acid changes” Eur. J. Biochem. 269:5950-5955 (2002); Persson, E., et al., “Augmented intrinsic activity of Factor VIIa by replacement of residues 305, 314, 337 and 374: Evidence of two unique mutational mechanisms of activity enhancement” Biochem. J. 379:497-503 (2004); and, Persson, E. “Variants of recombinant factor Vila with increased intrinsic activity” Semin. Hematol. 41 (Suppl. 1), 89-92 (2004). Studies have demonstrated improved procoagulant, antifibrinolytic and hemostasis properties in models of hemophilia A (see, e.g., Lisman et al. “Enhanced in vitro procoagulant and antifibrinolytic potential of superactive variants of recombinant factor VIIa in severe hemophilia A” J. Thromb. Haemost. 1:2175-2178 (2003); Tranholm et al. “Improved hemostasis with superactive analogs of factor VIIa in a mouse model of hemophilia A” Blood 102:3615-3620 (2003).

Active and inactive (zymogen-like) forms of serine proteases, e.g., FVIIa, exist in an equilibrium (see, e.g., Huber and Bode “Structural basis of the activation and action of trypsin” Acc. Chem. Res. 11: 114-122 (1978)), which is thought to favor the inactive state in the case of FVIIa (see, e.g., Higashi et al., “Molecular mechanism of tissue factor-mediated acceleration of factor VIIa activity” J. Biol. Chem. 271: 26569-26574 (1996)). Upon binding to TF, the equilibrium shifts such that the active form of FVIIa is now favored, leading to a catalytically competent enzyme.

Although not found in FVIIa, the zymogen-like form of the protease may even have some catalytic activity in some cases. See, e.g., Boose et al. “The single-chain form of tissue-type plasminogen activator has catalytic activity: Studies with a mutant enzyme that lacks the cleavage site” Biochemistry 28: 635-643 (1989); Lijnen et al. “Plasminogen activation with single-chain urokinase-type plasminogen activator (scu-PA). Studies with active site mutagenized plasminogen (Ser^{74→}Ala) and plasmin-resistant scu-PA (Lys⁵⁸⁴Glu)” J. Biol. Chem. 265: 5232-5236 (1990); Pasternak et al. “Activating a zymogen without proteolytic processing: mutation of Lys15 and Asn194 activates trypsinogen” Biochemistry 37: 16201-16210 (1998). In fact FVIIa activity is not optimal until it binds to its cofactor TF, shifting the equilibrium to the active form of FVIIa (see, e.g., Butenas et al. “Synthetic substrates for human factor VIIa and factor VIIa-tissue factor” Biochemistry 32: 6531-6538 (1993); Neuenschwander et al. Importance of substrate composition, pH and other variables on tissue factor enhancement of factor VIIa activity. Thromb. Haemost. 70: 970-977 (1993); and, Higashi et al. “Molecular mechanism of tissue factor-mediated acceleration of factor VIIa activity” J. Biol. Chem. 271: 26569-26574 (1996). In addition, certain residues on FVIIa that contact TF have major effects on TF-dependent activity (Dickinson et al. “Identification of surface residues mediating tissue factor binding and catalytic function of the serine protease factor VIIa” Proc. Natl. Acad. Sci. USA 93: 14379-14384 (1996); Dickinson and Ruf “Active site modification of factor VIIa affects interactions of the protease domain with tissue factor. J. Biol. Chem. 272: 19875-19879 (1997); and, Persson et al. “Substitution of aspartic acid for methionine-306 in factor VIIa abolishes the allosteric linkage between the active site and the binding interface with tissue factor” Biochemistry 40: 3251-3256 (2001).

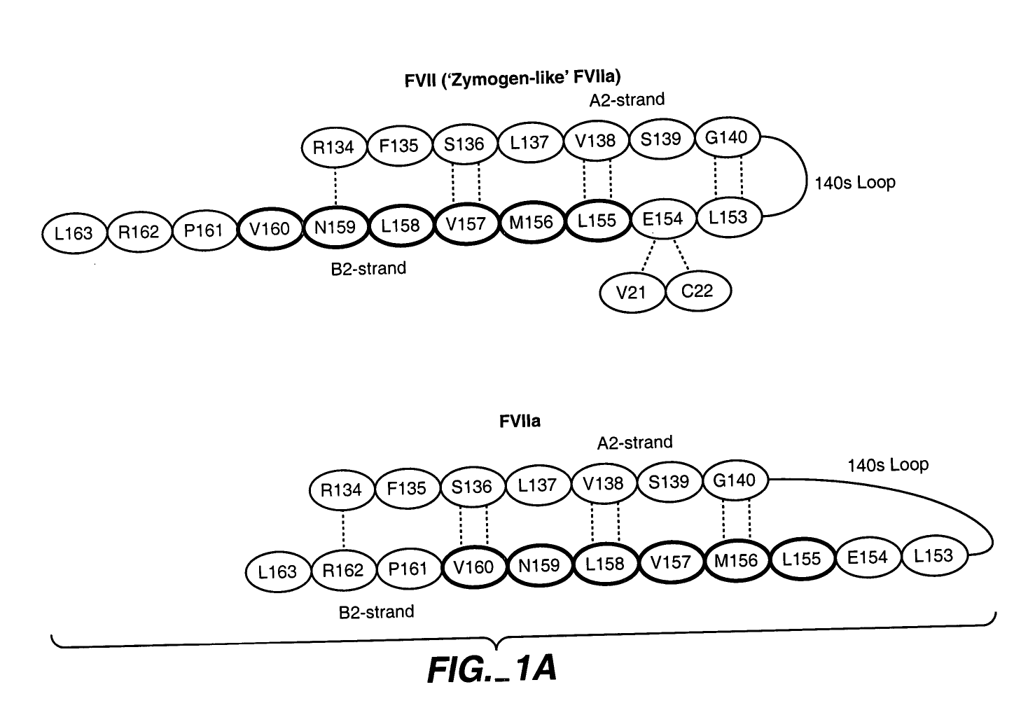

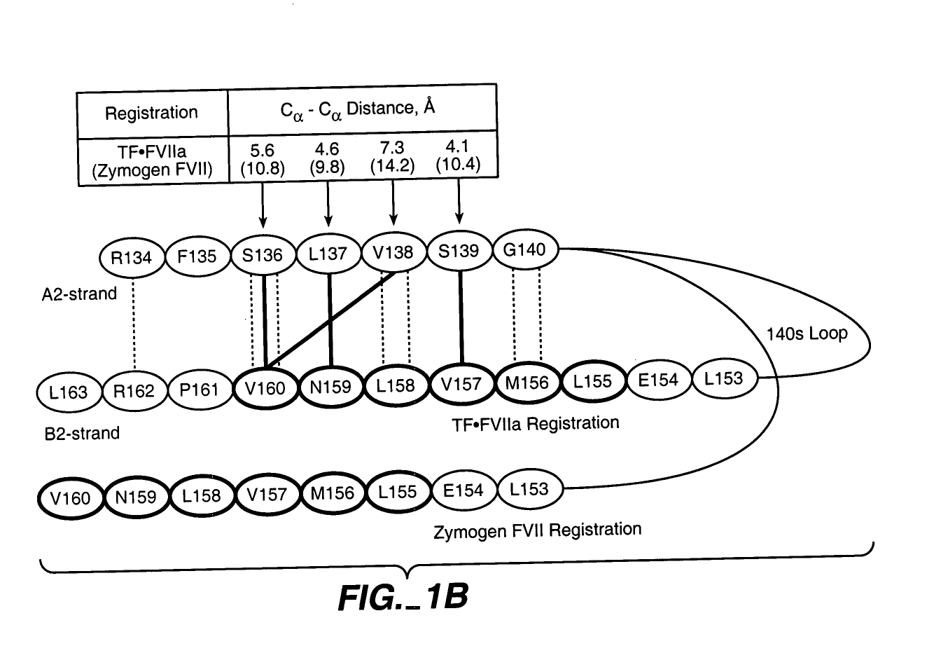

Comparisons between the zymogen FVII and TF•FVIIa structures have revealed typical conformational differences in the serine protease activation domain comprising the N-terminus and the c140s, c180s and c220s loops; (chymotrypsinogen numbering is used). See, e.g., Eigenbrot, C., et al., (2001) “The factor VII zymogen structure reveals reregistration of β-strands during activation” Structure 9:627-636 (2001); Eigenbrot, C. “Structure, function and activation of coagulation FVII” Curr. Protein Peptide Sci. 3:287-299 (2002); and, Eigenbrot, C. and Kirchhofer, D. (2002) “New insight into how tissue factor allosterically regulates factor VIIa” Trends Cardiovasc. Med. 12, 19-26 (2002). However a major change in the TF binding region of the protease domain is observed, due to a three-residue shift in β-strand B2 relative to strand A2 (FIG. 1, Panels A and B). Here residues Thr151 to Val160 were shifted toward the C-terminus relative to FVIIa, even though the main chain H-bond interactions between β-strands B2 and A2 are essentially identical in the zymogen FVII and TF•FVIIa structures. Upon inspection of this strand shift, zymogen-like or protease-like conformations could be imparted into FVIIa by engineering selectively placed cysteine residues into beta-strands A2 and B2 to form a disulfide bond and a locked conformation. The invention provides these and other variants of FVIIa, e.g., locked variants of FVIIa, and methods of producing and using such variants.

For example, a Factor VIIa (FVIIa) variant of the invention comprises an amino acid sequence derived from a mammalian FVIIa protein (e.g., a human FVIIa protein), where at least two amino acid residues are substituted with an amino acid (e.g., a cysteine amino acid, an unnatural amino acid or modified amino acid). In one embodiment, the two substituted amino acid residues can form a disulfide bond. The substitutions of the invention can lock A2-strand of FVIIa to B2-strand of FVIIa. Examples of pairs of substituted amino acids corresponding to a human amino acid residue pairs include, but are not limited to, e.g., S136 and V160, L137 and N159, V138 and V160, S139 and V157, F135 and N159, F135 and P161, V138 and L158, F135 and M156, and/or, V138 and L155.

FVIIa and variants of the invention can be prepared by a variety of methods well known in the art. Amino acid sequence variants of FVIIa can be prepared by mutations in the FVIIa DNA. See Recombinant Synthesis herein. For example, the FVIIa variants are prepared by site-directed mutagenesis of nucleotides in the DNA encoding the native FVIIa, thereby producing DNA encoding the variant, and thereafter expressing the DNA in recombinant cell culture.

Additional amino acid substitutions or amino acids deletions or insertions can also be present in a FVIIa variant of the invention, which can be made as described herein. For example, a FVIIa variant of the invention includes at least one additional, optionally, two or more, optionally, three or more, optionally four or more, etc., amino acid substitutions. Examples of additional amino acid substitutions which corresponds to a change in the human amino acid residue indicated include, but are not limited to, e.g., E17 (E154), V21 (V158), F135 (F278), S136 (S279), L137 (L280), V138 (V281), S139 (S282), E154 (E296), L155 (L297), M156 (M298), V157-(V299), L158 (L300), N159 (N301), V-160 (V302), L163 (L305), M164 (M306), D167 (D309), S170b (S314), K188 (K337) and/or F225 (F374). For example, the change in the human amino acid residue includes, but is not limited to, e.g., V21D (V158D), V21E (V158E), V21N (V158N), L1371 (L290I), E154V (E296V), E1541 (E296I), E154R (E296R), M156Q (M298Q), M156K (M298K), V157M (V299M), L163V (L305V), M164D (M306D), D167S (D309S), S170bE (S314E), K188A (K337A), and/or F225Y (F374Y). See, e.g., Petrovan & Ruf, J. Biol. Chem. 39:14457-14463 (2001); Tranholm et al., Blood 102:3615-3620 (2003); Lisman et al., J. Thromb. Haemostasis, 1:2175-2178 (2003); Persson et al., Biochem. J. 379:497-503 (2004); Persson et al., Semin. Hematol., 41 (1), Suppl. 1:89-92 (2004); Persson et al., J. Biol. Chem. 276(31):29195-29199 (2001); Persson & Olsen, Eur. J. Biochem., 269:5950-5955 (2002); Olsen et al., Biochemistry 43:14096-14103 (2004); U.S. Pat. No. 5,580,560; WO02/077218; WO03/027147; WO02/22776; EP 0370036B1; WO02/38162; and, WO03/029442. In one embodiment of the invention, a FVIIa variant of the invention lacks amino acid substitutions that create a cysteine pair at Cys 22 and Cys 157. See, e.g., Olsen et al., Biochemistry 43:14096-14103 (2004). FVIIa variants of the invention can also include additional mutations in the 99 loop and 170 loop; chymotrypsinogen numbering is used (see, e.g., Soejima, K., et al., “The 99 and 170 loop-modified factor Vila mutants show enhanced catalytic activity without tissue factor” J. Biol. Chem. 277:49027-49035 (2002)). Modifications in the Gla domain of FVIIa, e.g., to obtain higher membrane binding affinity, can also be present. See, e.g., Shah et al., PNAS USA 95:4229-4234 (1998), Harvey, S. B. et al. “Mutagenesis of the γ-carboxyglutamic acid domain of human factor VII to generate maximum enhancement of the membrane contact site” J. Biol. Chem. 278:8363-836 (2003); Nelsestuen, G. L. et al. “Elevated Function of Blood Clotting Factor VIIa Mutants That Have Enhanced Affinity for Membranes” J. Biol. Chem. 276:39825-39831(2001).

Activity of the FVIIa variants can be measured by a variety of methods well known in the art and those described herein. For example, FVII/FVIIa mediated or associated activity, or TF-FVIIa mediated or associated process, can be conveniently measured employing standard assays, such as those described in Roy, S., J. Biol. Chem. 266:4665-4668 (1991), O'Brien, D., et al., J. Clin. Invest. 82:206-212 (1988), Neuenschwander, et al. Thromb. Haemost. 70:970-977 (1993), Lee et al., Biochemistry 36:5607-5611 (1997), Kelly et al., J. Biol. Chem. 272:17467-17472 (1997), for the conversion of chromogenic substrates or Factor X to Factor Xa in the presence of Factor VII and other necessary reagents. See also, e.g., Persson & Olsen, “Assignment of molecular properties of a superactive coagulation factor VIIa variant to individual amino acid-changes” Eur. J. Biochem., 269:5950-5955 (2002); Persson et-al., “Substitution of Valine for Leucine 305 in Factor VIIa Increases the Intrinsic Enzymatic Activity” Journal of Biol. Chem. 276(31):29195-29199 (2001); Persson “Variants of Recombinant Factor VIIa with Increased Intrinsic Activity” Seminars in Hematology 41(1), Suppl. 1: 89-92 (2004); Persson et al., “Augmented intrinsic activity of Factor VIIa by replacement of residues 305, 314, 337 and 374: Evidence of two unique mutational mechanisms of activity enhancement” Biochem. J. 379:497-503 (2004); Petrovan, R. J. and Ruf, W. (2001) “Residue Met156 contributes to the labile enzyme conformation of coagulation factor VIIa” J. Biol. Chem. 276:6616-6620 (2001); Soejima, K., et al., “The 99 and 170 loop-modified factor VIIa mutants show enhanced catalytic activity without tissue factor” J. Biol. Chem. 277:4902749035 (2002); Kelley, R. F., et al., (1995) “Analysis of the factor VIIa binding site on human tissue factor: effects of tissue factor mutations on the kinetics and thermodynamics of binding” Biochemistry 34:10383-10392 (1995), etc. A FVIIa variant of the invention optionally includes an enhanced activity in the absence of tissue factor protein compared to a naturally occurring mammalian FVIIa protein. In certain embodiments, additional amino acid substitution(s) contributes to FVIIa variant activity.

Recombinant Synthesis

The invention includes isolated nucleic acids, preferably DNA, encoding variants described herein. DNAs encoding the variants of the invention can be prepared by a variety of methods known in the art. These methods include, but are not limited to, recombinant DNA techniques, such as site-specific mutagenesis (Kunkel et al., Methods Enzymol. 204:125-139 (1991); Carter, P., et al., Nucl. Acids. Res. 13:4331 (1986); Zoller, M. J., et al., Nucl. Acids Res. 10:6487 (1982)), cassette mutagenesis (Wells, J. A., et al., Gene 34:315 (1985)), restriction selection mutagenesis (Wells, J. A., et al., Philos. Trans, R. Soc. London, SerA 317, 415), and the like. See also, e.g., Sambrook, J. et al., Molecular Cloning (3rd ed.), Cold Spring Harbor Laboratory, N.Y., (2001); Ausbel, et al., Short Protocols in Molecular Biology, 5^{th} Edition, Current Protocols, (2002); and, Adelman et al., DNA, 2:183 (1983). Methods of producing a FVIIa variant are also included in the invention. For example, a method includes culturing the host cell with the DNA encoding a FVIIa variant of the invention, under condition suitable for expression of the FVIIa variant. The FVIIa variant can optionally be recovered from the culture medium.

An expression control sequence can be operably linked to the DNA molecule encoding a variant of the invention, and an expression vector, such as a plasmid, comprising the DNA molecule, where the control sequence is recognized by a host cell transformed with the vector. In general, plasmid vectors contain replication and control sequences which are derived from species compatible with the host cell. The vector ordinarily carries a replication site, as well as sequences which encode proteins that are capable of providing phenotypic selection in transformed cells.

Suitable host cells for expressing the DNA include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC No. 31,446); E. coli ₓ1776 (ATCC No. 31,537); E. coli strain W3110 (ATCC No. 27,325) and K5 772 (ATCC No. 53,635). The host cells referred to in this disclosure encompass cells in in vitro culture as well as cells that are within a host animal.

In addition to prokaryotes, eukaryotic organisms, such as yeasts, or cells derived from multicellular organisms can be used as host cells. For expression in yeast host cells, such as common baker's yeast or Saccharomyces cerevisiae, suitable vectors include episomally replicating vectors based on the 2-micron plasmid, integration vectors, and yeast artificial chromosome (YAC) vectors. Suitable host cells for expression also are derived from multicellular organisms. Examples of invertebrate cells include insect cells such as Drosophila S2 and Spodoptera Sf9, as well as plant cells. For expression in insect host cells, such as Sf9 cells, suitable vectors include baculoviral vectors. For expression in plant host cells, particularly dicotyledonous plant hosts, such as tobacco, suitable expression vectors include vectors derived from the Ti plasmid of Agrobacterium tumefaciens.

Examples of useful mammalian host cells include monkey kidney CV1 line transformed by SV40 (COS-7, ATCC CRL 1651); human embryonic kidney line (293 cells subcloned for growth in suspension culture, Graham et al., J. Gen Virol. 36:59 (1977)); baby hamster kidney cells (BHK, ATCC CCL 10); Chinese hamster ovary cells/-DHFR(CHO, Urlaub and Chasin, Proc. Natl. Acad. Sci. USA 77:4216 (1980)); DP12 cells (CHO K1 DUX B11 (DHFR-)), Lucas, B. K., et al., “High-level production of recombinant proteins in CHO cells using a dicistronic DHFR intron expression vector” Nucl. Acid Res. 24:1774-1779 (1996); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI38, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma cell line (Hep G2).

For expression in prokaryotic hosts, suitable vectors include pBR322 (ATCC No. 37,017), phGH107 (ATCC No. 40,011), pBO475, pS0132, pRIT5, any vector in the pRIT20 or pRIT30 series (Nilsson and Abrahmsen, Meth. Enzymol. 185:144-161 (1990)), pRIT2T, pKK233-2, pDR540 and pPL-lambda. Prokaryotic host cells containing the expression vectors of the invention include E. coli K12 strain 294 (ATCC NO. 31,446), E. coli strain JM101 (Messing et al., Nucl. Acid Res. 9:309 (1981)), E. coli strain B, E. coli strain _{X}1776 (ATCC No. 31,537), E. coli c600 (Appleyard, Genetics 39:440 (1954)), E. coli W3110 (F-, gamma-, prototrophic, ATCC No. 27,325), E. coli strain 27C7 (W3110, tonA, phoA E15, (argF-lac)169, ptr3, degP41, ompT, kan.sup.r) (U.S. Pat. No. 5,288,931, ATCC No. 55,244), Bacillus subtilis, Salmonella typhimurium, Serratia marcesans, and Pseudomonas species.

For expression in mammalian host cells, useful vectors include vectors derived from SV40, vectors derived from cytomegalovirus such as the pRK vectors, including pRK5, pRK7, pRKCT31 (Suva et al., Science 237:893-896 (1987); EP 307,247 (Mar. 15, 1989), EP 278,776 (Aug. 17, 1988); Roberge M. et al. “A Novel Exosite on Coagulation Factor VIIa and its Molecular Interactions with a New Class of Peptide Inhibitors” Biochemistry 40:9522-9531 (2001)) vectors derived from vaccinia viruses or other pox viruses, and retroviral vectors such as vectors derived from Moloney's murine leukemia virus (MoMLV). pCMV.DI.tPA and pCMV.PD5.IRES-GFP can be used as an mammalian expression vector. See, e.g., Lucas, B. K., et al., “High-level production of recombinant proteins in CHO cells using a dicistronic DHFR intron expression vector” Nucl. Acid Res. 24:1774-1779 (1996); and Example 1, herein.

Optionally, the DNA encoding the FVIIa variant of interest is operably linked to a secretory leader sequence resulting in secretion of the expression product by the host cell into the culture medium. Examples of secretory leader sequences include stII, ecotin, lamB, herpes GD, lpp, alkaline phosphatase, invertase, MIP.5 and alpha factor. Also suitable for use herein is the 36 amino acid leader sequence of protein A (Abrahmsen et al., EMBO J. 4:3901 (1985)).

Host cells are transfected and preferably transformed with the above-described expression or cloning vectors of this invention and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending upon the host cell used, transformation is done using standard techniques appropriate to such cells. The calcium treatment employing calcium chloride, as described in Sambrook et al., Molecular Cloning, 3rd ed. (Cold Spring Harbor Laboratory, New York, 2001), is generally used for prokaryotes or other cells that contain substantial cell-wall barriers. Infection with Agrobacterium tumefaciens is used for transformation of certain plant cells, as described by Shaw et al., Gene 23:315 (1983) and WO 89/05859, published Jun. 29, 1989. For mammalian cells without such cell walls, the calcium phosphate precipitation method described in Sambrook et al., supra, is typically used. General aspects of mammalian cell host system transformations have been described by Axel in U.S. Pat. No. 4,399,216, issued Aug. 16, 1983. Transformations into yeast are typically carried out according to the method of Van Solingen et al., J. Bact. 130:946 (1977) and Hsiao et al., Proc. Natl. Acad. Sci. USA 76:3829 (1979). However, other methods for introducing DNA into cells such as by nuclear injection, electroporation, or by protoplast fusion may also be used.

Other vectors can be constructed using standard techniques by combining the relevant traits of the vectors described above. Relevant traits include the promoter, the ribosome binding site, the gene of interest or gene fusion (the Z domain of protein A and gene of interest and a linker), the antibiotic resistance markers, and the appropriate origins of replication.

A variation on the above procedures contemplates the use of gene fusions, wherein the gene encoding the desired peptide is associated, in the vector, with a gene encoding another protein or a fragment of another protein. This results in the desired peptide being produced by the host cell as a fusion with another protein or peptide. The “other” protein or peptide is often a protein or peptide which can be secreted by the cell, making it possible to isolate and purify the desired peptide from the culture medium and eliminating the necessity of destroying the host cells which arises when the desired peptide remains inside the cell. Alternatively, the fusion protein can be expressed intracellularly. It is useful to use fusion proteins that are highly expressed.

The use of gene fusions, though not essential, can facilitate the expression of heterologous peptides in insect cells as well as the subsequent purification of those gene products. Protein A fusions are often used because the binding of protein A, or more specifically the Z domain of protein A, to IgG provides an “affinity handle” for the purification of the fused protein. For example, a DNA sequence encoding the desired peptide ligand can be fused by site-directed mutagenesis to the gene for a consensus domain of protein A known as the Z domain (Nilsson et al., Protein Engineering 1: 107-113 (1987)). After expression and secretion the fusion protein can be enzymatically cleaved to yield free peptide which can be purified from the enzymatic mix (see, e.g., Varadarajan et al., Proc. Natl. Acad. Sci USA 82:5681-5684 (1985); Castellanos-Serra et al., FEBS Letters 378:171-176 (1996); Nilsson et al., J. Biotechnol. 48:241-250 (1996)).

Fusion proteins can be cleaved using chemicals, such as cyanogen bromide, which cleaves at a methionine, or hydroxylamine, which cleaves between an Asn and Gly residue. Using standard recombinant DNA methodology, the nucleotide base pairs encoding these amino acids may be inserted just prior to the 5′ end of the gene encoding the desired peptide.

Alternatively, one can employ proteolytic cleavage of fusion protein. Carter, in Protein Purification: From Molecular Mechanisms to Large-Scale Processes, Ladisch et al., eds., Ch. 13, pp. 181-193 (American Chemical Society Symposium Series No. 427, 1990).

Proteases such as enterokinase, Factor Xa, thrombin, and subtilisin or its mutants, and a number of others have been successfully used to cleave fusion proteins. Trypsin cleavage is discussed generally in Nilsson et al., J. Biotech. 48:241 (1996) and Smith et al., Methods Mol. Biol. 32:289 (1994). Typically, a peptide linker that is amenable to cleavage by the protease used is inserted between the “other” protein (e.g., the Z domain of protein A) and the desired peptide. Using recombinant DNA methodology, the nucleotide base pairs encoding the linker are inserted between the genes or gene fragments coding for the other proteins. Proteolytic cleavage of the partially purified fusion protein containing the correct linker can then be carried out on either the native fusion protein, or the reduced or denatured fusion protein.

The variant may or may not be properly folded when expressed, e.g., as a fusion protein. Also, the specific peptide linker containing the cleavage site may or may not be accessible to the protease. These factors determine whether the fusion protein must be denatured and refolded, and if so, whether these procedures are employed before or after cleavage.

When denaturing and refolding are needed, typically the peptide is treated with a chaotrope, such a guanidine HCl, and is then treated with a redox buffer, containing, for example, reduced and oxidized dithiothreitol or glutathione at the appropriate ratios, pH, and temperature, such that the peptide is refolded to its native structure.

Disulfide Linked Variants

The locked formation of the FVIIa variants of the invention can be achieved by the formation, for example, of a disulfide bond between Cys residues. Residues capable of forming a disulfide bond include for example Cys, Pen, Mpr, and Mpp and its 2-amino group-containing equivalents. The locked formation of the FVIIa variants of the invention can also be achieved by the formation of a lactam linkage. Residues capable of forming a lactam bridge include, for example, Asp, Glu, Lys, Orn, -diaminobutyric acid, diaminoacetic acid, aminobenzoic acid and mercaptobenzoic acid. The compounds herein can be locked, for example, via a lactam bond which can utilize the side chain group of a non-adjacent residue to form a covalent attachment to the N-terminus amino group of Cys or other amino acid. Lactams can also be formed between side chains of two non adjacent residues, for example a Lys in the appropriate position in strand A2 and an Asp, Asn, Glu or Gln in the appropriate position in strand in B2. Alternative bridge structures also can be used to locked the compounds of the invention, including, for example, unnatural amino acids, modified amino acids, peptides and peptidomimetics, etc., which can cyclize via S—S, CH₂—S, CH₂—O—CH₂, lactam ester or other linkages.

FVIIa variants of the invention can be made by recombinant methods as herein and then locked by any convenient method used in the formation of disulfide linkages. For example, FVIIa variants can be recovered with sulfhydryls in reduced form, dissolved in a dilute solution wherein the intramolecular cysteine concentration exceeds the intermolecular cysteine concentration in order to optimize intramolecular disulfide bond formation, such as a polypeptide concentration of 25 mM to 1 μM, or 500 μM to 1 μM, or 25 μM to 1 μM, and then oxidized by exposing the free sulfhydryl groups to a mild oxidizing agent that is sufficient to generate intramolecular disulfide bonds, e.g., molecular oxygen with or without catalysts such as metal cations, potassium ferricyanide, sodium tetrathionate, etc. The variants can be locked as described in, e.g., Pelton et al., J. Med. Chem. 29:2370-2375 (1986). FVIIa disulfide formations can be analyzed by methods known by one of skill in the art, including, but not limited to, e.g., SDS-PAGE under reducing and non-reducing conditions, mass spectrometry under reducing and non-reducing conditions, etc.

Diagnostic Methods and Compositions

This invention encompasses methods of screening compounds to identify those that mimic or enhance the FVIIa variants (agonists) or prevent or inhibit the effect of the FVIIa variants (antagonists). Screening assays for antagonists are designed to identify compounds that bind or complex with the FVIIa variant described herein, or otherwise interfere with the interaction of the encoded polypeptides with other cellular proteins. Such screening assays will include assays amenable to high-throughput screening of chemical libraries, making them particularly suitable for identifying small molecule candidates. The assays can be performed in a variety of formats, including protein-protein binding assays, biochemical screening assays, immunoassays, and cell-based assays, which are well characterized in the art.

In certain embodiments, the variants of the invention are non-covalently adsorbed or covalently bound to a macromolecule, such as a solid support. It will be appreciated that the invention encompasses both macromolecules complexed with the variants. In general, the solid support is an inert matrix, such as a polymeric gel, comprising a three-dimensional structure, lattice or network of a material. Almost any macromolecule, synthetic or natural, can form a gel in a suitable liquid when suitably cross-linked with a bifunctional reagent. In certain aspects, the macromolecule selected is convenient for use in affinity chromatography. Most chromatographic matrices used for affinity chromatography are xerogels. Such gels shrink on drying to a compact solid comprising only the gel matrix. When the dried xerogel is resuspended in the liquid, the gel matrix-imbibes liquid, swells and returns to the gel state. Xerogels suitable for use herein include polymeric gels, such as cellulose, cross-linked dextrans (e.g., Sepharose), agarose, cross-linked agarose, polyacrylamide gels, and polyacrylamide-agarose gels.

Alternatively, aerogels can be used for affinity chromatography. These gels do not shrink on drying but merely allow penetration of the surrounding air. When the dry gel is exposed to liquid, the latter displaces the air in the gel. Aerogels suitable for use herein include porous glass and ceramic gels.

Also encompassed herein are the variants of the invention coupled to derivatized gels wherein the derivative moieties facilitate the coupling of the variants to the gel matrix and avoid steric hindrance in affinity chromatography. Alternatively, spacer arms can be interposed between the gel matrix and the variant for similar benefits.

Pharmaceutical Compositions

Pharmaceutical compositions which comprise the compounds, including the FVIIa variants of the invention, may be formulated and delivered or administered in a manner best suited to the particular FVII/FVIIa mediated disease or disorder being treated, including formulations suitable for parental, topical, oral, local, aerosol or transdermal administration or delivery of the compounds.

In certain embodiments of the invention, suitable compositions of the invention comprise any of the compounds described herein along with a pharmaceutically acceptable carrier, the nature of the carrier differing with the mode of administration delivery or use, for example, in oral administration, usually using a solid carrier and in i.v. administration, a liquid salt solution carrier. Alternatively, the variant may be provided in a formulation that would allow for the variant to slowly elute from a formulation, e.g., a sustained release formation, providing both local and systemic events associated with inducing coagulation. Patches and bandages are also available, e.g., for topical administration of a FVIIa variant of the invention.

The compositions of the invention include pharmaceutically acceptable components that are compatible with the subject and the compound of the invention. These generally include suspensions, solutions and elixirs, and most especially biological buffers, such as phosphate buffered saline, saline, Dulbecco's Media, and the like. Aerosols may also be used, or carriers such as starches, sugars, microcrystalline cellulose, diluents, granulating agents, lubricants, binders, disintegrating agents, and the like (in the case of oral solid preparations, such as powders, capsules, and tablets).

As used herein, the term “pharmaceutically acceptable” generally means approved by a regulatory agency of the Federal or a state government or listed in the U.S. Pharmacopeia or other generally recognized pharmacopeia for use in animals, and more particularly in humans.

The formulation of choice can be made using a variety of the aforementioned buffers, or even excipients including, for example, pharmaceutical grades of mannitol, lactose, starch, magnesium stearate, sodium saccharin cellulose, magnesium carbonate, and the like. “PEGylation” of the compositions may be achieved using techniques known to the art (see for example International Patent Publication No. WO92/16555, U.S. Pat. No. 5,122,614 to Enzon, and International Patent Publication No. WO92/00748). Oral compositions can be taken in the form of solutions, suspensions, tablets, pills, capsules, sustained release formations, powders, etc.

Phospholipids and combinations of phospholipids can also be present. For example, in certain embodiments, FVIIa variants of the invention are administered with phospholipid compositions. Such phopholipid compositions are typically formulated to form phospholipids vesicle and/or liposome compositions, as are generally known in the art. As described, suitable phospholipids for use in the vesicle/liposome compositions of the invention include those which contain fatty acids having twelve to twenty carbon atoms; said fatty acids may be either saturated or unsaturated. Preferred phospholipids for use according to the invention include phosphatidylcholine (PC), phosphatidylethanolamine (PE), phosphatidylglycerol (PG) and phosphatidylserine (PS). These phospholipids may come from any natural source and the phospholipids, as such, may be comprised of molecules with differing fatty acids. Phospholipid mixtures comprising phospholipids from different sources may be used. For example, PC, PG and PE may be obtained from egg yolk; PS may be obtained from animal brain and spinal chord. These phospholipids may come from synthetic sources as well. The phospholipids are conveniently combined in the appropriate ratios to provide the phospholipid mixture for use in preparing the composition of the invention. See, e.g., Butenas, S., et al., “Influence of factor VIIa and phospholipids on coagulation in “acquired” hemophilia” Arterioscler. Thromb. Vasc. Biol. 23:123-129 (2003).

The preparation of vesicles and/or liposomes is generally well known and has been previously described. Exemplary methods for preparation of vesicles and/or liposomes include, but are not limited to, e.g., Butenas, S., et al., “Influence of factor VIIa and phospholipids on coagulation in “acquired” hemophilia” Arterioscler. Thromb. Vasc. Biol. 23:123-129 (2003); U.S. Pat. No. 5,104,661; Lopez-Berenstein et al., J. Infect. Dis., 151:704-710 (1985); Lopez-Berenstein, Antimicrob. Agents Chemother., 31:675-678 (1987); Lopez-Berenstein et al., J. Infect. Dis., 150:278-283 (1984); and, Mehta et al., Biochem. Biophys. Acta, 770:230-234 (1984).

Liposomes with enhanced circulation time may also be prepared as described in U.S. Pat. No. 5,013,556. Thus, in one embodiment, the invention contemplates the administration of the FVIIa variants of the invention with phospholipids vesicles and/or liposomes.

Therapeutic Methods

The compounds of the invention can be used therapeutically to alter coagulation. The alteration of coagulation is desirable in indications where there are bleeding disorders and induction of the coagulation would be beneficial.

Thus, the invention encompasses a method for altering (e.g., inducing) coagulation in a mammal comprising administering to the mammal an effective amount of the variant of the invention. An effective amount of the compound of the invention is predetermined to achieve the desired effect. The amount to be employed therapeutically will vary depending upon therapeutic objectives, the routes of administration and the condition being treated. Accordingly, the dosages to be administered are sufficient to induce coagulation in the subject being treated.

The therapeutic effectiveness is measured by an improvement in one or more symptoms associated with the coagulation disorders. Such therapeutically effective dosages can be determined by the skilled artisan and will vary depending upon the age, sex and condition of the subject being treated. Suitable dosage ranges for systemic administration are typically between about 1 μg/kg to up to 100 mg/kg or more and depend upon the route of administration. According to the invention, a preferred therapeutic dosage is between about 1 μg/kg body weight and about 5 mg/kg body weight. For example, suitable regimens include-intravenous injection or infusion sufficient to maintain concentration in the blood in the ranges specified for the therapy contemplated.

The conditions characterized by abnormal coagulation include, but are not limited to, e.g., thrombotic or coagulopathic related diseases or disorders, Hemophilia A (FVIII deficiency), Hemophilia B (factor IX deficiency), Hemophilia C (factor XI deficiency), hemophilia with inhibitors and acquired inhibitors of factors VIII and X, Christmas disease (Factor IX deficiency), Stuart factor disease (factor X deficiency), SPCA (serum prothrombin conversion accelerator) deficiency (factor VII deficiency), clotting disorders due to Vitamin K deficiencies, liver disease, liver transplantation, renal failure, intractable bleeding, fibrinogen deficiencies (liver disease, disseminated intravascular coagulation (DIC), L-asparaginase therapy, rattlesnake bites), clotting factor deficiencies, circulating anticoagulants (e.g., in the case of lymphoma, SLE, idiopathic), massive transfusion (e.g., dilutional coagulopathy), anticoagulation-induced bleeding, surgery, platelet disorders, thrombocytopenia, thrombasthenia, von Willebrand disease, Bernard-Soulier syndrome, vascular disease, inflammatory responses, bone marrow problems, bone marrow transplantation, pregnancy bleeding disorders, traumatic bleeding, and other bleeding disorders, etc.

FVIIa variants of the invention can also be administered in combination with other agents used for bleeding disorders. The conventional dosage range of an agent used for bleeding disorders is the daily dosage used in therapy and is readily available to the treating physician. See, e.g., Physicians Desk Reference 2003, 57^{th} Edition, Thomson Healthcare, publisher. Bleeding disorder agents include, but are not limited to, e.g., cryoprecipitate, desmopressin acetate (DDAVP), recombinant FVIIa (e.g., NovoSeven®), an agent, e.g., a recombinant or purified factor, that is a replacement for a missing or reduced (e.g., due genetics, or to antibody production against the factor) clotting factor, e.g., VII, VIII and/or IX, Vitamin K supplementation, platelets, fresh-frozen plasma, ε-aminocaproic acid (Amicar), aprotinin (Trasylol), etc. See, e.g., Lisman & DeGroot, Journal of Thrombosis and Haemostasis, 1:1138-1139 (2003); Midathada et al., Am. J. Clin. Pathol., 121:124-137 (2004); and, Lawson & Murphy, Seminars in Hematology 41(1), Suppl. 1:55-64 (2004). Tissue factor and tissue factor variants can also be administered with the FVIIa variants of the invention. With DIC (disseminated intravascular coagulation), immediate treatment may be crucial and complex. Since DIC involves both clotting and bleeding throughout the body, treatment may involve platelet and clotting factor transfusions as well as heparin or other anticoagulant therapy.

The term combination as used herein includes a single dosage form containing at least the FVIIa variant of the invention and at least one agent to induce coagulation or anticoagulation (e.g., as in the case of DIC). The term is also meant to include multiple dosage forms where a FVIIa variant of the invention is administered separately from the other agent(s) either concurrently or sequentially by two or more separate administration. Typically, these combinations and compositions work (e.g., either additively or synergistically) to induce coagulation resulting in clot formation.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1

### FVIIa Locked Variants

We expressed 7 FVIIa-like variants and 2 zymogen-like variants and purified them by TF affinity chromatography. Mass spectrometry analysis of tryptic peptides from the FVIIa variants confirmed the new disulfide bond formation. Kinetic analysis of amidolytic activity using several chromogenic substrates revealed that several of the FVIIa-like disulfide locked variants alone had increases in specific activity compared to wildtype FVIIa. FVIIa variants 136:160 and 138:160 with substrate S-2765, had 670- and 330-fold increases, respectively. Several disulfide locked variants no longer required TF as a cofactor for maximal activity in amidolytic assays. Activity was also enhanced for the FVIIa-like disulfide locked variants in the presence of soluble TF compared to wildtype. For example, activity was enhanced for the 136:160 and 138:160 variants in the presence of TF, e.g., 20- and 12-fold respectively compared to wildtype. In the presence of relipidated TF, mutants 136:160 and 137:159 also had a ca. 3-fold increase in their Vₘₐₓ/Kₘ values for FX activation.

Materials and Methods

Mutant Design: The designed disulfide links were engineered by seeking one residue each in β-strands A2 and B2, where a disulfide might reasonably form without large changes to the direction of the vectors from Cα to Cβ, i.e., without changing the direction in which the side chain was projected. See, e.g., FIG. 1, Panels A and B. Residues Phe135, Ser136, Leu137, Val138, and Ser139 from α-strand A2 and Val157, Asn159 and Val160 from β-strand B2 are the middle section of main chain-main chain H-bonds between the two β-strands in both zymogen and enzyme structures. Disulfide links from each of these positions in A2 to positions in B2 were designed, and, because of the close correspondence in backbone conformation of B2 in zymogen and enzyme, the partner position in B2 could be either to a zymogen position or an enzyme position, which is shifted by 3 residues. Thus, a cysteine at position 135 could link to position 156 in the zymogen registration, or to position 159 in the enzyme registration, and one would predict relatively poor activity for a 135:156 disulfide and relatively good activity for a 135:159 disulfide. Similarly, potential disulfide links were conceived from 136 to 157 (FVII) or 160 (FVIIa), from 137 to 156 (FVII) or 159 (FVIIa), from 138 to either 155 or 157 (FVII) or either 158 or 160 (FVIIa), and from 139 to 154 (FVII) or 157 (FVIIa). The designs were evaluated visually for steric conflicts and judged to have accessible conformations consistent with formation of engineered covalent links.

Mutagenesis and Construction of Plasmids: The wildtype FVII expression plasmid pRKCT31 (Roberge et al. (2001) supra) was used as a starting template for Kunkel mutagenesis (Kunkel, T. A., et al., “Rapid and efficient site-specific mutagenesis without phenotypic selection” Methods Enzymol. 154:367-382 (1987)) to generate the various mutant-encoding plasmids. The following primers were used in combination to introduce the indicated mutations: VII-F135C (5′-GCT GAC CAA TGA GCA GCG CAC GAA GGC-3′), VII-S136C (5′-GCT GAC CAA GCA GAA GCG CAC-3′), VII-L137C (5′-GCC GCT GAC GCA TGA GAA GCG-3′), VII-V138C (5′-GCC CCA GCC GCT GCA CAA TGA GAA GCG-3′), VII-S139C (5′-GCC CCA GCC GCA GAC CAA TGA-3′), VII-L155C (5′-GTT GAG GAC CAT GCA CTC CAG GGC CGT-3′), VII-M156C (5′-CAC GTT GAG GAC GCA GAG CTC CAG GGC-3′), VII-V157C (5′-CAC GTT GAG GCA CAT GAG CTC-3′), VII-L158C (5′-GGG CAC GTT GCA GAC CAT GAG-3′), VII-N159C (5′-CCG GGG CAC GCA GAG GAC CAT-3′), VII-V160C (5′-CAG CCG GGG GCA GTT GAG GAC-3′) and VII-P161C (5′-CAT CAG CCG GCA CAC GTT GAG-3′).

Two primers were used in one Kunkel mutagenesis reaction to introduce the nine different FVII variants. The entire cDNAs encoding the double-mutants were verified by DNA sequencing to exclude the presence of unwanted mutations.

The cDNAs encoding the various FVII double mutants were cloned into the EcoR1 and HindIII sites of the mammalian expression vector pCMV.PD5.IRES-GFP, which was derived from vector pCMV.DI.tPA (Lucas, B. K., et al., “High-level production of recombinant proteins in CHO cells using a dicistronic DHFR intron expression vector” Nucl. Acid Res. 24:1774-1779 (1996)) by introducing IRES-GFP downstream of the target gene. Plasmids were prepared by using the QIAprep spin miniprep kits (Qiagen, Valencia, Calif.).

Cell Culture, Transfection, Selection and Expression: 1 to 1.5×10⁶ DP12 cells (CHO K1 DUX B11 (DHFR⁻) (Lucas, B. K., et al., (1996) supra) were seeded on 100 mm dish in 10 ml DP12 media (F12/DMEM low glucose media containing 10% FBS (Sigma, St. Louis Mo.), 1% glutamine, 100 μg/ml penicillin, 250 μg/ml streptomycin (Invitrogen, Carlsbad, Calif.) 1 mM HEPES pH 7.2 and thymidine 5 μg/ml (GHT)) 24 h before transfection. 1.2 ml transfection media (HG DMEM without FBS) were mixed with 36 μl FuGENE 6 (Roche Applied Science, Indianapolis, Ind.) in a sterile tube and incubated for 5 min at room temperature. 12 μg pCMV.PD5.IRES-GFP expression plasmid encoding FVII mutant was added and incubated for 15 min at RT. FuGENE 6/plasmid mixture was added dropwise to DP12 cells and incubated for 48 h at 37° C. Transfected cells were split after 48 h and maintained in DP12 media containing 10 μg/ml puromycin to select for stable transfectants.

Stable transfectants were then sorted by FACS on a Beckmann Coulter Epics Elite Flow Cytometer for the top 5% in fluorescence intensity due to the GFP reporter. Cells were maintained for expression in DP 12 media including 10 μg/ml puromycin. FVII variants were expressed from stable cell pools in serum-free media containing trace elements, 10 μg/ml human insulin and 6 μg/ml vitamin K (Aquamephyton, Merck, Whitehouse Station, N.J.) at 32° C. Medium containing secreted FVII variant was harvested after 7 days of incubation.

Purification of FVIIa Variants: An FVII affinity column was prepared by immobilizing 13 mg of soluble tissue factor (sTF) (Kelley, R. F., et al., “Analysis of the factor VIIa binding site on human tissue factor: effects of tissue factor mutations on the kinetics and thermodynamics of binding” Biochemistry 34:10383-10392 (1995)) on a 1 ml HiTrap NHS-activated HP column (Amersham Biosciences, Piscataway, N.J.) following the manufacturer's instruction. Harvested tissue culture media was sterile filtered and brought to 5 mM CaCl₂ and 20 mM Tris pH 8 before loading at 1 ml/min onto the immobilized sTF column, previously equilibrated with wash buffer (20 mM Tris pH 8, 5 mM CaCl₂, 135 mM NaCl and 2 mM benzamidine). The column was washed with 10 column volumes of wash buffer and eluted with 5 column volumes of 20 mM Tris pH 8, 150 mM NaCl, 10 mM EDTA and 2 mM benzamidine. The eluate was concentrated and subjected to size exclusion on a Superdex 200 Tricor column (Amersham Biosciences, Piscataway, N.J.) for further purification in running buffer (20 mM Tris pH 8, 300 mM NaCl, 10 mM EDTA) at a flow rate of 0.5 ml/min. Fractions containing FVII variants were pooled and concentrated.

Activation of FVII Variants: FVII variants were mixed with 1/10 (w/w) biotinylated FXa (Roche Applied Science, Indianapolis, Ind.) and brought to 1.5 ml final volume in 50 mM Tris pH 8, 100 mM NaCl, 5 mM CaCl₂. Following incubation for 4 h at room temperature, biotinylated FXa was removed with Streptavidin beads as suggested in the manufacturer's protocol.

FVIIa Mutant Characterization: All FVIIa variants were analyzed by SDS-PAGE in nonreduced or reduced form; samples were reduced by addition of 1 μl of 14.3 M β-mercaptoethanol (Sigma, St. Louis, Mo.) to sample and boiling for 3 min prior to SDS-PAGE analysis on a 4-20% Tris-Glycine Novex gel followed by staining with Coomassie Blue. Protein concentrations were determined by amino acid analysis and OD₂₈₀ with an extinction coefficient of (1.34 g/l)⁻¹×cm⁻¹. Amino acid analysis confirmed the calculated extinction coefficient was accurate to determine the protein concentration by OD₂₈₀. All FVIIa variants were active site titrated using the Kunitz domain inhibitor TF7I-C, quantified by active site titrated trypsin, as described to determine the concentration of active sites (see, e.g., Dennis and Lazarus “Kunitz domain inhibitors of tissue factor-factor VIIa. I. Potent inhibitors selected from libraries by phage display” J. Biol. Chem. 269: 22129-22136 (1994); and, Seymour et al. “Ecotin is a potent anticoagulant and reversible tight-binding inhibitor of factor Xa” Biochemistry 33: 3949-3958 (1994)).

Mass Spectrometry of FVIIa Variants: Mass spectrometry was used to confirm the presence of the additionally introduced disulfide bond. 100 μg of FVII (mutant or wildtype) was incubated with 5-fold molar excess of iodoacetamide (Sigma, St. Louis, Mo.) in 50 mM ammonium bicarbonate pH 7.5 for 15 min at room temperature in the dark in order to alkylate all free cysteines. After alkylation, FVII was digested with 2.5 μg trypsin (Promega, Madison Wis.) in 50% acetonitrile at 37° C. overnight. The entire digest mixture was analyzed in the oxidized and reduced state (addition of β-mercaptoethanol) by mass spectrometry to identify peptide masses that correlated with the disulfide-linked peptides from β-strands A2 and B2.

Nonreduced peptides were analyzed by orthogonal MALDI-TOF MS (QSTAR XL; Applied Biosystems, Foster City, Calif.) and capillary HPLC electrospray ion trap tandem mass spectrometry. MALDI samples were prepared by 1:1 mixture with alpha-cyano-4-hydroxycinnamic acid (Agilent Technologies, Wilmington, Del.) and 1 μL applied to the sample probe and dried under ambient conditions. For LC-MS analysis, sample aliquots were injected onto 75 μm id Picofrit capillary columns (New Objective Inc., Woburn, Mass.), packed with 9 cm of C18 resin (5 μm, Michrom Bioresources, Auburn, Calif.). Peptides were eluted directly into the microelectrospray source of an LCQ Deca XP-plus mass spectrometer with a gradient of 0-40% acetonitrile in 0.1% acetic acid, 0.005% TFA at a flow rate of 200 nL/min. The mass spectrometer performed MS and MS/MS scans in a data-dependent experiment; full mass range MS scans were followed by collision-induced dissociation (CID) scans of the three most intense ions detected. A dynamic exclusion list prevented any precursor ion from being subjected to CID more than twice. The disulfide-linked peptides of interest were identified by examination of the mass spectral data. Reconstructed ion chromatograms were plotted for the doubly and triply charged ions of each anticipated peptide dimer. The corresponding CID spectra were then interpreted, matching observed fragment ions to those predicted for each peptide.

Disulfide-linked peptides were reduced for 1 hour at 37° C. with 1 mM DTT. The reduced peptide mixture (1 μl) was diluted with 1 μl of 2,5-DHB matrix (2,5-dihydroxybenzoic acid, Agilent), spotted onto a stainless steel maldi plate and allowed to air dry at room temperature. MALDI-TOF mass spectrometry was performed on a Voyager-DE STR instrument (Applied Biosystems) operated in reflection mode with delayed extraction.

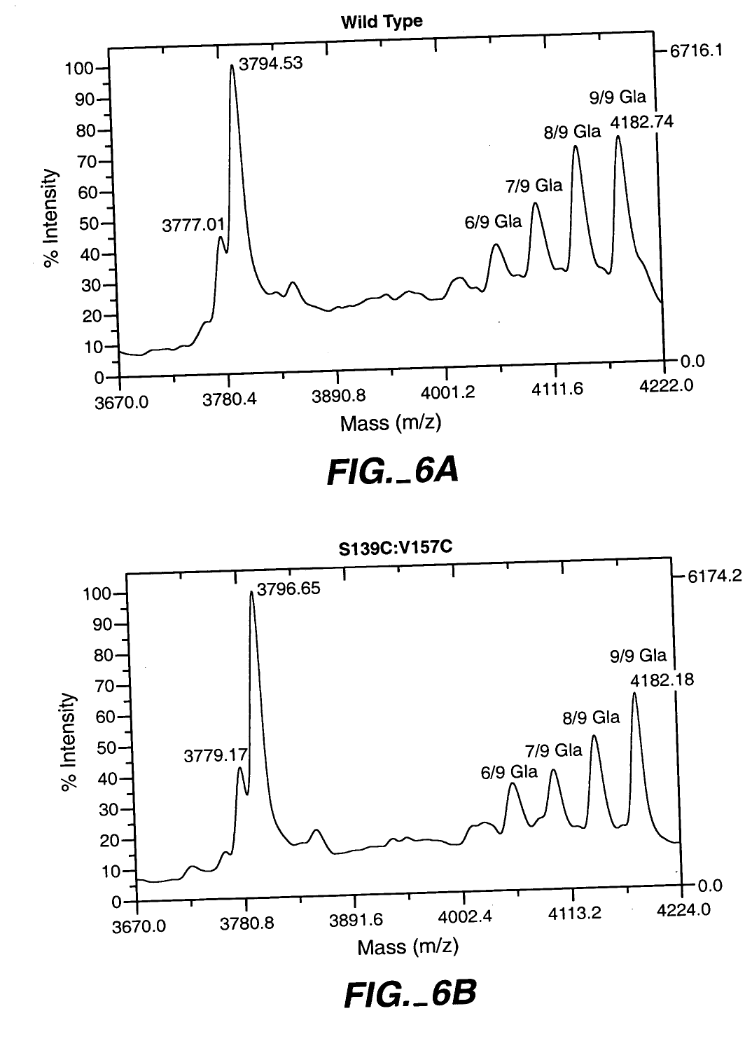

The extent to which Gla domain glutamic acid residues were post-translationally modified to γ-carboxyglutamic acids was determined by MALDI-TOF mass spectrometry as described above with the following differences. Digestion with trypsin was carried out after reduction of disulfides with 10 mM DTT and was stopped after 2 h. The MALDI matrix used in this experiment was a saturated solution of 5-methoxysalicylic acid (Tokyo Kagei Kogyo Co, LTD., Tokyo, Japan) in 60% acetonitrile/0.1% TFA. MALDI-TOF mass spectrometry was performed in the linear mode of the Voyager-DE STR with delayed extraction. See FIG. 6, Panels A and B. Other variants displayed the same pattern.

FVIIa Amidolytic Activity Assay: The amidolytic activity of FVIIa and the FVIIa variants were measured using chromogenic substrates Chromozym tPA; N-methylsulphonyl-D-Phe-L-Gly-L-Arg-pNA (Roche Applied Science, Indianapolis, Ind.), S-2288; H-D-Ile-L-Pro-L-Arg-pNA, S-2765; Z-D-Arg-L-Gly-L-Arg-pNA where Z is a benzoyl group (DiaPharma, West Chester, Ohio) and Spectrozyme fXa; methoxycarbonyl-D-cyclohexylglycyl-L-Gly-L-Arg pNA (American Diagnostica, Stamford, Conn.). FVIIa and FVIIa variants (30 nM) in the absence and presence of sTF (10 nM FVIIa, 250 nM sTF for S-2288 and Chromozym t-PA; 30 nM FVIIa, 100 nM sTF for S-2765 and Spectrozyme fXa) were incubated with varying concentrations of chromogenic substrates (ranging from 10 mM to 2 μM) in a final volume of 100 μl containing 100 mM Hepes pH 7.8, 140 mM NaCl, 0.1% PEG-8000, 0.02% Tween-20 and 5 mM CaCl₂. The absorbance of released pNA was monitored at 405 nm on a SpectraMax Plus³⁸⁴ microplate reader (Molecular Devices, Sunnyvale, Calif.) at ambient temperature. Conversion to μmol/min was calculated using μmol pNA/min=(0.0417)×(mOD₄₀₅/min); the conversion factor was determined with a standard curve of pNA in 100 μl of the same buffer. See Table 3. Initial rate data were fitted to the Michaelis-Menten equation using Kaleidagraph (Synergy Software, Reading, Pa.) and Kₘ and Vₘₐₓ values were determined from the averages of 3 independent determinations.

TABLE 3
Conversion of mOD/min to μmoL/min:
Active site titration Correction

| | | | Active site | |
|---|---|---|---|---|
| | Active | mOD to | Conversion | |
| | site Con- | μmol Con- | Factor and | |
| | version | version | mOD to μmol | |
| | factor | Factor | Conversion | |
| | Multiply | Multiply | Factor | |
| | by | by | Multiply by | 50 nM Enzyme |
| WT | 0.87 | .0417 | 0.036279 | WT = 57.5 nM |
| 136:160 | 3.65 | .0417 | 0.152205 | J (136:160) = 13.7 nM |
| 137:159 | 2.22 | .0417 | 0.092574 | K (137:159) = |
| | | | | 22.5 nM |
| 138:160 | 2.07 | .0417 | 0.086319 | M (138:160) = |
| | | | | 24.1 nM |
| 139:157 | 0.96 | .0417 | 0.040032 | N (139:157) = |
| | | | | 52.2 nM |

μmol/min = 0.0417 × mOD₄₀₅/min

FVIIa Proteolytic Activation Assay: FVIIa or FVIIa mutant (1 nM) and 0.4 nM relipidated TF₁₋₂₄₃ in phosphotidylcholine/phosphotidylserine (PC/PS) vesicles, 70/30 was mixed with varying concentrations of FX (1000 nM to 0.5 nM) in a final volume of 100 μL containing 20 mM Hepes pH 7.4, 150 mM NaCl, 5 mM CaCl₂ and 0.5 mg/ml BSA. After a 3 min incubation the reaction was quenched with 40 mM EDTA; controls were run to determine that rates were linear with different quench times indicating that substrate depletion did not occur.

Spectrozyme fXa was added to yield a final concentration and volume of 0.5 mM and 200 μl, respectively. The amount of generated FXa was determined by monitoring the OD₄₀₅/min on a SpectraMax Plus³⁸⁴ microplate reader at ambient temperature. Initial rate data were fitted to the Michaelis-Menten equation using Kaleidagraph and Kₘ and Vₘₐₓ determined from the averages of 3 independent determinations.

Due to the high Kₘ value for FX in the absence of negatively charged phospholipid vesicles and TF, the activity of FVIIa and variants were measured at one fixed FX concentration as described above. The proteolytic activity was tested with 100 nM FVIIa alone, 10 nM FVIIa with 100 nM sTF and 10 nM FVIIa with 0.5 mM PC/PS (70/30) phospholipid vesicles. S-2765 was used as a chromogenic substrate to determine the amount of FXa generated. Initial rates were obtained as above and used to calculate the relative activity of the FVIIa variants compared to wildtype. Background activity of FVIIa variants towards S-2765 was subtracted prior to comparison.

Binding of FVIIa Variants to sTF by Surface Plasmon Resonance: The effects of the mutations in FVIIa upon binding to sTF were determined by surface plasmon resonance measurements on a Biacore 3000 instrument (Biacore, Piscataway, N.J.). Soluble TF was immobilized on a CM5 sensor chip surface by coupling through free amino groups. The carboxylated dextran matrix was first activated with a mixture of N-hydroxysuccinimide (NHS) and 1-ethyl-3-(1-dimethylaminopropyl)-carbodiimide (EDC) using a protocol provided by the manufacturer. A 20 μl injection of 50 μg/ml sTF in 10 mM sodium acetate pH 4 at a flow rate of 5 μl/min resulted in the immobilization of 750 resonance units above baseline. Unreacted NHS was blocked by injection of 35 μl 1 M ethanolamine. The affinity of FVIIa variants for sTF was calculated from the binding kinetics of binding to immobilized sTF. For a 1:1 binding interaction, A+B⇄AB, where K_{D}=k_{off}/kₒₙ. The dissociation rate constant (k_{off}) was determined by analyzing the response curve observed upon return to buffer flow for 6 minutes after saturation with various concentrations of FVIIa. Association rate constants (kₛ) were calculated by using a series of seven FVIIa concentrations ranging from 6.125 nM to 400 nM in 2-fold increments. 100 μl of each sample was injected and kₒₙ was determined from the concentration dependence of kₛ. A flow rate of 5 μl/min was employed for all kinetics measurements with buffer containing 20 mM Tris pH 7.5, 100 mM NaCl, 5 mM CaCl₂, 0.05% Tween 20 and 0.01% NaN₃. The sensor chip surface was regenerated by elution of bound VIIa with an injection of 50 mM EDTA. Kinetic constants were determined by nonlinear regression analysis using software supplied by the manufacturer.

Clotting Activity in Human FVII Deficient Plasma: FVIIa and FVIIa variants were diluted to a concentration of 5 μg/ml directly into FVII deficient plasma from three different donors—lots 523b1 and N2521 (George King Bio-Medical, (Overland Park, Kans.) and lot 707/045 (American Diagnostica, Stamford, Conn.), all having <1% FVII. Each stock was further diluted with additional FVII deficient plasma to cover a final concentration range of 5 μg/ml to 5 μg/ml FVIIa in the plasma. In an ACL 6000 coagulometer (Beckman Coulter, Fullerton, Calif.), one part plasma±FVIIa was mixed with 2 parts Innovin® (Dade, Miami, Fla.) prothrombin time reagent (recombinant human tissue factor with phospholipids and CaCl₂). Clot formation was detected optically and time to clotting measured. Clotting time (seconds) was compared to the mean clotting time of FVII-deficient plasma alone, which had a clotting time of ca. 90 seconds, and plotted as the fractional clotting time versus FVIIa concentration.

Results and Discussion

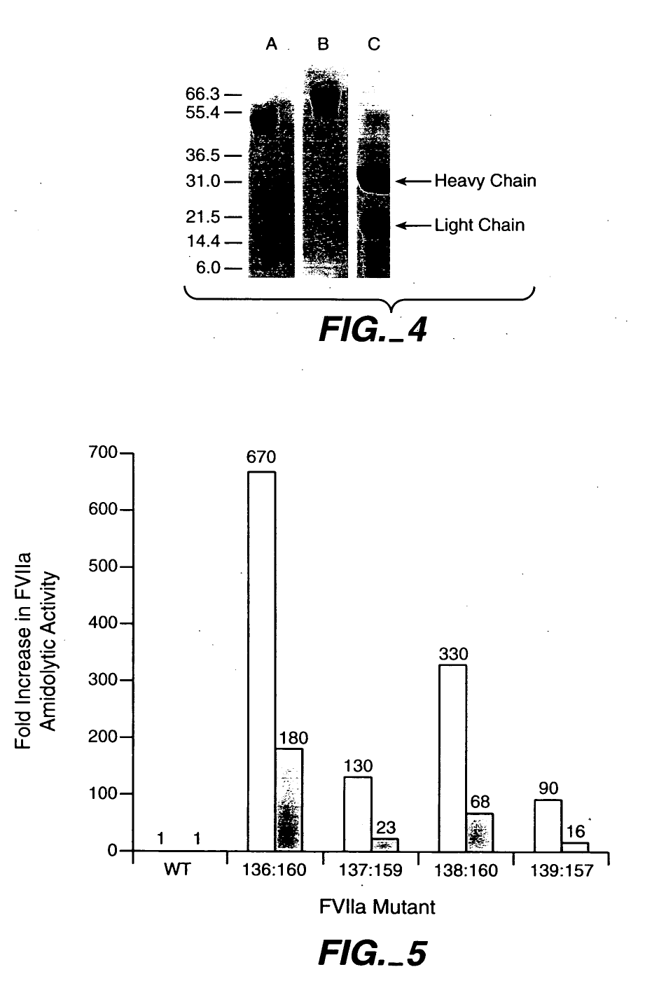

Expression and Purification: The use of the specifically engineered mammalian expression vector pCMV.PD5.IRES-GFP facilitated and accelerated the generation of stable pools of cells expressing high yields of FVII variants (ca. 1 μg/ml culture media). Comparing this yield with previously transient transfections, an improvement of 100- to 1000-fold was found. All variants were initially purified out of serum free media using a sTF affinity column, thus verifying that the different FVII variants were properly folded and contained a competent TF binding site. Since the high affinity binding of FVII to TF is highly Ca²⁺ dependent, mild elution conditions were applicable by the addition of 10 mM EDTA and the absence of Ca²⁺ in the elution buffer. In order to ensure a high degree of purity, further contaminants were removed by size exclusion. All variants were analyzed by SDS-PAGE under reducing and non-reducing conditions. Variants were purified as zymogens indicated by a single band under both reducing and non-reducing conditions. For example, FIG. 4 illustrates a gel for mutant 139:157 (FIG. 4, lanes A and B). All variants were expressed as zymogen and remained intact. All other variants basically gave the same results from SDS-PAGE analysis under these conditions.

Characterization of Disulfide Locked FVII variants: The introduction of 2 new cysteines into wildtype FVII was confirmed. Verification that the specific cysteines paired as a disulfide was also determined, because of the presence of 12 other disulfide bonds in FVII. Based on the primary sequence of wildtype FVII, we found that the two β-strands A2 (residues 134-140) and B2 (residues 153-162) containing the cysteine mutations were flanked by arginines—R134, R147 and R162. A tryptic digest should result in the formation of two individual peptides, which if cross-linked due to disulfide bond formation would be detectable as one mass under non-reducing conditions or two individual masses under reducing conditions. The mass of the disulfide-linked tryptic peptides for all variants before and after reduction with β-mercaptoethanol was clearly identified by MS analysis (Table 4) which indicated that the correct disulfide bonds were indeed present. A detailed analysis of the mass spectrometry data did not reveal any evidence for alternate structures, i.e. no unpaired Cys-containing peptides were observed in the non-reduced sample, nor were there peaks at masses corresponding to mispaired disulfide linked peptides.

TABLE 4
Mass spectrometry analysis of FVIIa tryptic peptide digest

| | | | Tryptic Peptide Digest | | | | | |
|---|---|---|---|---|---|---|---|---|
| | | | Mass | | | | | |
| | | | (amu) | | | | | |
| | | | Nonreduced | | Reduced | | | |
| FVIIa | FVII | | Phe135-Arg162 | | Phe135-Arg147 | | Gly149-Arg162^{a} | |
| Mutant | state | Abbrev. | Calcd. | Obs. | Calcd. | Obs. | Calcd. | Obs. |
| WT | | | — | — | 1476.77 | 1476.7 | 1482.82 | 1482.7 |
| S136C:V160C | TF•FVII | 136:160 | 2977.50 | 2977.3 | 1492.75 | 1492.8 | 1486.76 | 1486.8 |
| | a-like | | | | | | | |
| L137C:N159C | TF•FVII | 137:159 | 2936.48 | 2936.2 | 1466.70 | 1466.6 | 1471.79 | 1471.6 |
| | a-like | | | | | | | |
| V138C:V160C | TF•FVII | 138:160 | 2965.47 | 2966.3^{b} | 1480.71 | 1480.7 | 1486.76 | 1486.8 |
| | a-like | | | | | | | |
| S139C:V157C | TF•FVII | 139:157 | 2977.50 | 2977.0^{b} | 1492.75 | 1492.6 | 1486.76 | 1486.7 |
| | a-like | | | | | | | |
| F135C:N159C | TF•FVII | 135:159 | 2902.49 | 2902.9 | 1432.71 | 1432.5 | 1471.79 | 1471.6 |
| | a-like | | | | | | | |
| F135C:P161C | TF•FVII | 135:161 | 2919.48 | 2920.1 | 1432.71 | 1432.7 | 1488.78 | 1488.8 |
| | a-like | | | | | | | |
| V138C:L158C | TF•FVII | 138:158 | 2951.45 | 2951.6 | 1480.71 | 1480.7 | 1472.75 | 1472.8 |
| | a-like | | | | | | | |
| F135C:M156C | FVII- | 135:156 | 2885.49 | 2885.7 | 1432.71 | 1432.8 | 1454.79 | 1454.8 |
| | like | | | | | | | |
| V138C:L155C | FVII- | 138:155 | 2951.45 | 2951.9 | 1480.71 | 1480.8 | 1472.75 | 1472.9 |
| | like | | | | | | | |

^{a}refers to tryptic peptide Gly149-Arg162; there is no residue 148 in chymotrypsinogen numbering.
^{b}Average mass, from LC-ESI-ion trap MS; calculated masses and all other measured masses are monoisotopic, from MALDI-TOF or MALDI-QTOF MS.

Kinetic Analysis of Amidolytic Activity of FVIIa Disulfide Locked Variants: The FVII disulfide locked variants were then activated to FVIIa using biotinylated FXa followed by its removal with Strepavidin beads. Activation was confirmed by SDS-PAGE in nonreduced or reduced form. Activation was confirmed by reduced SDS-PAGE where loss of a single FVII band at ˜60 kDa resulted in the appearance of separate heavy and light chains for FVIIa; a representative gel is shown for mutant 139:157 (FIG. 4, lane C). The enzymes were then tested for their amidolytic and proteolytic activities. Characterization of the amidolytic activity of the FVIIa variants is dependent on conditions of the assay, such as salt concentration, pH, solubitity of the chromogenic substrate and additives such as detergents, PEG or BSA (Neuenschwander, P. F., et al., “Importance of substrate composition, pH and other variables on tissue factor enhancement of factor VIIa activity” Thromb. Haemost. 70:970-977 (1993)).

We initially screened variants for amidolytic activity in the absence and presence of sTF under different conditions. Several variants showed activities similar to or higher than wildtype. For example, since Chromozym t-PA was reported to be the most active chromogenic substrate for both FVIIa alone and the TF•FVIIa complex, we initially tested all variants for amidolytic activity in the absence and presence of sTF with 10 and 2 mM Chromozym t-PA, respectively. See Table 5.

TABLE 5
Activity of FVIIa variants in amidolytic activity assays with Chromozyme t-PA
as substrate^{a}

| | 100 nM FVIIa | | 10 nM FVIIa | | | |
|---|---|---|---|---|---|---|
| | 2 mM | | 500 nM sTF | | | |
| | substrate | Fold | 2 mM substrate | Fold | 30 nM FVIIa | Fold |
| FVIIa | Avg | increase | Avg | increase | 10 mM substrate | increase |
| Mutant | mOD₄₀₅/min | (mutant/wt) | mOD₄₀₅/min | (mutant/wt) | Avg mOD₄₀₅/min | (mutant/wt) |
| FVIIa | 41.3 | 1.0 | 29.9 | 1.1 | 6.1 | 1.0 |
| WT | | | | | | |
| 135:156 | 37.1 | 0.9 | 5.0 | 0.2 | 7.7 | 1.3 |
| 138:155 | 45.4 | 1.1 | 5.9 | 0.2 | 8.3 | 1.4 |
| 135:159 | 9.4 | 0.2 | 24.6 | 0.8 | 1.9 | 0.3 |
| 135:161 | 39.1 | 0.9 | 7.9 | 0.3 | 7.1 | 1.2 |
| 136:160 | 77.9 | 2.2 | 40.1 | 0.5 | 13.1 | 2.2 |
| 137:159 | 34.7 | 0.8 | 88.9 | 3.0 | 5.0 | 0.8 |
| 138:158 | 34.5 | 0.8 | 4.1 | 0.1 | 5.7 | 0.9 |
| 138:160 | 50.8 | 1.2 | 11.3 | 0.4 | 9.9 | 1.6 |
| 139:157 | 64.2 | 1.6 | 56.4 | 1.9 | 11.0 | 1.8 |

^{a}Data obtained from the average of tripicate independent determinations.

Variants 136:160, 137:159, 138:160 and 139:157 showed activities similar to or higher than wildtype.

In order to characterize the kinetics of the selected variants, Kₘ and Vₘₐₓ values were determined for amidolytic activity with a variety of chromogenic substrates (see Table 6), including S-2765 and Spectrozyme fXa, Chromozym t-PA, and S-2288 (Table 7).

In the presence of sTF, variants 136:160 and 138:160 had 20.3- and 12.0-fold respective increases in S-2765 specific activity compared to wildtype due to altered Kₘ and Vₘₐₓ values. The same variants also had 8.8- and 4.0-fold increase in specific activity with Spectrozyme fXa. However, the activity for these variants was greatly reduced using Chromozym t-PA and S-2288 as substrates (Table 7). In general, changes in activity for FVIIa variants 137:159 and 139:157 were more moderate with all substrates.

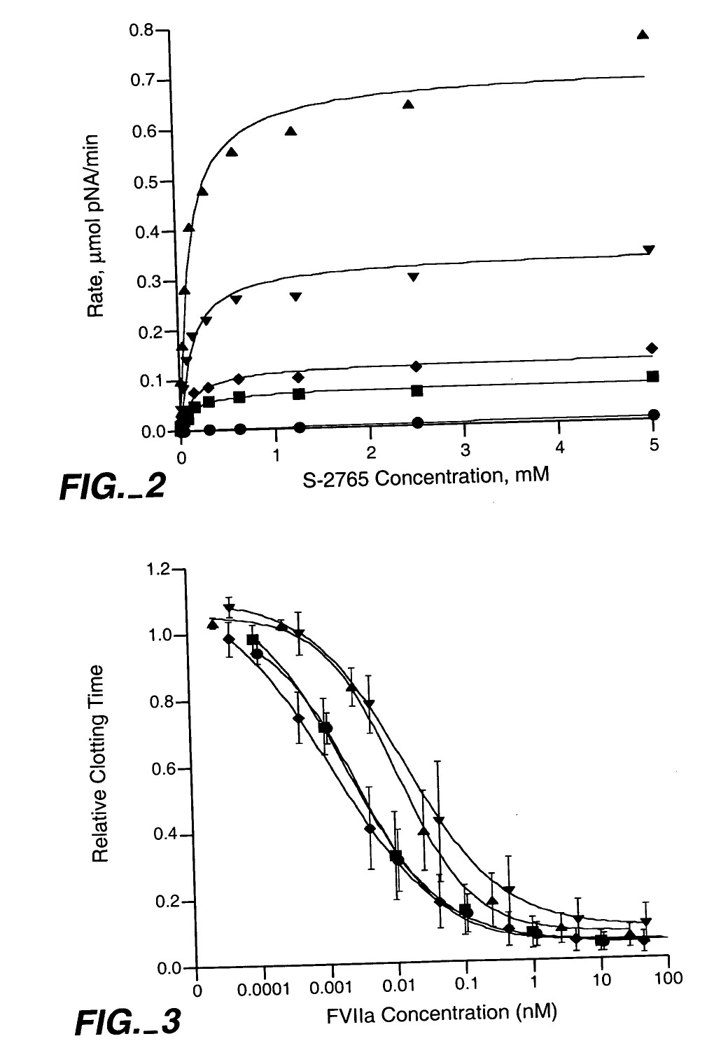

Further kinetic analysis of all four FVIIa variants in the absence of sTF was carried out with S-2765 and Spectrozyme fXa substrates; FVIIa variants alone with the other chromogenic substrates had activities too low to determine accurate kinetic constants. In the absence of sTF, all FVIIa variants exhibited significantly enhanced specific activity compared to wildtype (Table 8). A representative Michaelis-Menten plot is shown for variants in the absence of sTF with S-2765 in FIG. 2. FIG. 2 illustrates the kinetics of FVIIa variants with the peptide substrate S2765 to measure amidolytic activity. Representative individual kinetic analysis for amidolytic activity of S2765 with 30 nM FVIIa mutant is shown. Data for the Michaelis Menten plot were fit to a hyperbolic equation using Kaleidagraph from which values for Kₘ and Vₘₐₓ were derived. Triplicate independent determinations were performed. Variants 136:160 and 138:160 showed the strongest enhancement in specific amidolytic activity, having a 670- and 330-fold increase in S-2765 specific activity, respectively, due to favorable changes in both Kₘ and Vₘₐₓ (FIG. 5; Table 8). Similar trends were observed with 136:160 and 138:160 using Spectrozyme fXa as a substrate where 180- and 68-fold respective increases were determined, primarily due to the increase in Vₘₐₓ (FIG. 5; Table 8).

The effect of sTF for S-2765 and Spectrozyme fXa with a given mutant was assessed by comparing kinetic parameters in its presence and absence as a ratio (Table 8). Accordingly, sTF has a moderately large effect as a cofactor for wildtype, ranging from 18- to 30-fold increase in activity, having effects in both Kₘ and Vₘₐₓ, in reasonable agreement with previously published data (see, e.g., Neuenschwander et al. Importance of substrate composition, pH and other variables on tissue factor enhancement of factor VIIa activity. Thromb. Haemost. 70: 970-977 (1993); and, Neuenschwander and Morrissey Roles of the membrane-interactive regions of Factor VIIa and tissue factor. J. Biol. Chem. 269: 8007-8013 (1994)). Most notable is the complete lack of a TF-dependent rate enhancement for all of the variants except 137:159 with Spectrozyme fXa where only a 2-fold effect was observed. The role of TF as a cofactor for FVIIa has been eliminated.

Overall the data demostrates that locking the A2 and B2 beta-strand registration in FVIIa, which would restrict the reregistration of these strands in their zymogen-like and protease-like forms, can lead to variants with significantly higher enzymatic activity, especially in the absence of TF. Substrate-specificity across the various FVIIa disulfide locked variants varied. We note that all substrates contain Arg in P1 position. Substrates S-2765, Spectrozyme fXa, and Chromozym t-PA have a Gly at the P2 position, whereas S-2288, which is relatively poor substrate for the variants in the absence of sTF, has a Pro at P2. The different substrate effects are most likely due to changes at the P3 position where S-2765, Spectrozyme fXa, Chromozym t-PA and S-2288 have D-Arg, D-cyclohexylglycyl, D-Phe and D-Ile, respectively. See, Table 6.

TABLE 6
Chromogenic substrates

| Substrate | P3 | P2 | P1 |
|---|---|---|---|
| S-2765 | D-Arg | Gly | Arg |
| S-2288 | D-Ile | Pro | Arg |
| Spectrozyme fXa | D-CHG^{a} | Gly | Arg |
| Chromozym t-PA | D-F | Gly | Arg |

^{a}CHG refers to cyclohexylglycyl Chromozym t-PA - N-Methylsulfonyl-D-Phe-Gly-Arg-4-nitranilide acetate S-2288 - H-D-Ile-Pro-Arg-pNA

TABLE 7
Kinetic parameters for amidolytic activity of FVIIa
disulfide locked variants with different chromogenic substrates
in the presence of sTF

| | | sTF•FVIIa | |
|---|---|---|---|
| | Kₘ | Vₘₐₓ | Vₘₐₓ/Kₘ |
| FVIIa Mutant | mM^{a} | μmol/min^{a} | (Fold Mut/WT) |
| S-2765 | | | |
| WT | 2.0 ± 0.03 | 0.42 ± 0.01 | 0.21 (1.0) |
| 136:160 | 0.20 ± 0.04 | 0.85 ± 0.09 | 4.26 (20.3) |
| 137:159 | 2.2 ± 0.6 | 1.08 ± 0.10 | 0.49 (2.3) |
| 138:160 | 0.13 ± 0.02 | 0.33 ± 0.01 | 2.52 (12.0) |
| 139:157 | 1.3 ± 0.2 | 0.28 ± 0.03 | 0.21 (1.0) |
| Spectrozyme fXa | | | |
| WT | 1.5 ± 0.1 | 0.75 ± 0.06 | 0.50 (1.0) |
| 136:160 | 0.16 ± 0.2 | 0.70 ± 0.05 | 4.38 (8.8) |
| 137:159 | 1.9 ± 0.4 | 2.67 ± 0.42 | 1.40 (2.8) |
| 138:160 | 0.15 ± 0.01 | 0.30 ± 0.02 | 2.01 (4.0) |
| 139:157 | 0.71 ± 0.3 | 0.31 ± 0.08 | 0.44 (0.9) |
| Chromozym t-PA | | | |
| WT | 1.6 ± 0.03 | 1.16 ± 0.03 | 0.73 (1.0) |
| 136:160 | 1.3 ± 0.2 | 0.43 ± 0.03 | 0.33 (0.4) |
| 137:159 | 2.2 ± 0.15 | 4.51 ± 0.18 | 2.05 (2.8) |
| 138:160 | 1.0 ± 0.3 | 0.16 ± 0.02 | 0.16 (0.2) |
| 139:157 | 5.6 ± 0.6 | 2.13 ± 0.10 | 0.38 (0.5) |
| S-2288 | | | |
| WT | 1.9 ± 0.2 | 0.81 ± 0.02 | 0.43 (1.0) |
| 136:160 | 3.1 ± 0.4 | 0.40 ± 0.03 | 0.13 (0.3) |
| 137:159 | 2.5 ± 0.4 | 3.18 ± 0.16 | 1.27 (3.0) |
| 138:160 | 4.2 ± 0.8 | 0.18 ± 0.03 | 0.04 (0.1) |
| 139:157 | 5.0 ± 0.9 | 1.27 ± 0.19 | 0.25 (0.6) |

^{a}represents the average of at least 3 independent determinations; errors are reported at the standard deviation to the mean.

TABLE 8
Kinetic parameters for amidolytic activity and TF dependence of FVIIa
disulfide locked variants with different chromogenic substrates

| | | | | | sTF Ratio^{a} | |
|---|---|---|---|---|---|---|
| | | FVIIa | | | (sTF•FVIIa/ | |
| FVIIa | Kₘ | Vₘₐₓ | Vₘₐₓ/Kₘ | | FVIIa) | |
| Mutant | mM^{b} | μmol/min^{b} | (Fold Mut/WT) | Kₘ | Vₘₐₓ | Vₘₐₓ/Kₘ |
| S-2765 | | | | | | |
| WT | 1.2 ± 0.7 | 0.0087 ± 0.004 | 0.0073 (1) | (1.7) | (49) | (30) |
| 136:160 | 0.15 ± 0.02 | 0.733 ± 0.03 | 4.9 (670) | (1.3) | (1.2) | (0.9) |
| 137:159 | 0.14 ± 0.0 | 0.13 ± 0.01 | 0.93 (130) | (16) | (8.4) | (0.5) |
| 138:160 | 0.13 ± 0.2 | 0.32 ± 0.03 | 2.4 (330) | (1.0) | (1.0) | (1.0) |
| 139:157 | 0.11 ± 0.1 | 0.076 ± 0.004 | 0.68 (90) | (12) | (3.6) | (0.3) |
| Spectrozyme | | | | | | |
| fXa | | | | | | |
| WT | 0.34 ± 0.07 | 0.094 ± 0.03 | 0.028 (1) | (4.4) | (80) | (18) |
| 136:160 | 0.13 ± 0.02 | 0.64 ± 0.03 | 4.9 (180) | (1.2) | (1.1) | (0.9) |
| 137:159 | 0.26 ± 0.02 | 0.17 ± 0.01 | 0.64 (23) | (7.3) | (16) | (2.2) |
| 138:160 | 0.17 ± 0.02 | 0.32 ± 0.02 | 1.9 (68) | (0.9) | (0.9) | (1.1) |
| 139:157 | 0.21 ± 0.4 | 0.092 ± 0.03 | 0.44 (16) | (3.4) | (3.4) | (1.0) |

^{a}the sTF ratio (in parenthesis) refers to the fold effect of sTF on the indicated kinetic constant; i.e. the constant for FVIIa in the presence of sTF divided by that in the absence of sTF.
^{b}represents the average of at least 3 independent determinations; errors are reported at the standard deviation to the mean.

TF Binding to FVIIa Disulfide Locked Variants: The effects of the mutations in FVIIa upon binding to sTF were determined by surface plasmon resonance (Table 9). In this assay, wildtype FVIIa had a Kd of 5.2 nM, in good agreement with data previously reported (Kelley, R. F. et al “Similar Molecular Interactions of Factor VII and Factor VIIa with the Tissue Factor Region that Allosterically Regulates Enzyme Activity” Biochemistry 43, 1223-1229 (2004)). All of the disulfide locked variants were somewhat impaired in their ability to by sTF, mutant M-138:160 having the most significant loss in binding of 12-fold. All variants had moderately slower association rates and slightly faster dissociation rates.

TABLE 9
Kinetics and binding interactions of FVIIa Variants
with sTF by surface plasmon resonance

| | kₒₙ × 10⁻⁵, M⁻¹s⁻¹ | k_{off} × 10³, s⁻¹ | K_{D}, nM |
|---|---|---|---|
| FVIIa Mutant | (Fold Mut/WT) | (Fold Mut/WT) | (Fold Mut/WT) |
| WT | 2.3 (1.0) | 1.2 (1.0) | 5.2 (1.0) |
| 136:160 | 0.39 (0.17) | 1.5 (1.3) | 38 (7.3) |
| 137:159 | 0.80 (0.35) | 1.7 (1.4) | 21 (4.0) |
| 138:160 | 0.49 (0.21) | 3.1 (2.6) | 63 (12) |
| 139:157 | 1.6 (0.70) | 2.7 (2.3) | 17 (3.3) |

Effects of FVIIa Disulfide Locked Variants in FX Activation and Clotting Assays: We were able to determine Kₘ and Vₘₐₓ values for proteolytic activation of human FX by FVIIa and FVIIa variants carried out in the presence of relipidated TF in negatively charged phospholipid vesicles PC/PS with a ratio of 70/30 (Table 10). Variants 136:160 and 137:159 showed a ca. 3-fold improvement over wildtype in their Vₘₐₓ/Kₘ values due to both lower Kₘ and higher Vₘₐₓ values; the other variants were essentially the same as wildtype. The absence of either lipid vesicles or TF or both dramatically increases the Kₘ for FX. In the absence of relipidated TF, the relative proteolytic activity of 10 nM FVIIa mutant or wildtype at a fixed concentration of 1 μM FX with 0.5 mM PC/PS (70/30) phospholipid vesicles, was as follows: WT, 100%; 136:160, 67%; 137:159, 79%; 138:160, 40%; 139:157, 24%. In the absence of phospholipid vesicles, the relative proteolytic activity of 10 nM FVIIa variants or wildtype at a fixed concentration of 1 μM FX with 100 nM sTF, was as follows: WT, 100%; 136:160, 71%; 137:159, 110%; 138:160, 36%; 139:157, 82%.

TABLE 10
FX activation by relipidated TF•FVIIa^{a}

| | Kₘ | Vₘₐₓ | Vₘₐₓ/Kₘ |
|---|---|---|---|
| FVIIa Mutant | nM | mOD₄₀₅/min | (fold Vₘₐₓ/Kₘ) |
| WT | 130 ± 9 | 600 ± 23 | 4.6 (1.0) |
| 136:160 | 50 ± 10 | 765 ± 18 | 15.3 (3.3) |
| 137:159 | 87 ± 5 | 1130 ± 33 | 13.0 (2.8) |
| 138:160 | 55 ± 1 | 205 ± 6 | 3.7 (0.8) |
| 139:157 | 110 ± 7 | 480 ± 19 | 4.4 (0.9) |

^{a}expedments were carried out using 0.4 nM relipidated TF and 1 nM FVIIa (normalized by active site titration) in triplicate and values are reported as the average ± standard deviation.

The disulfide-locked variants were further analyzed in a TF-dependent clotting assay with FVII deficient plasma, using varying concentrations of each mutant and wildtype FVIIa. FIG. 3 illustrates the relative TF-dependent clotting in FVII deficient plasma. Relative clotting times were normalized to the clotting time in FVII deficient plasma. The average data from 3 independent determinations were fit by a four parameter fit using Kaleidagraph; the error as standard deviation is shown. In the presence of TF, variants 137:159 and 139:157 had similar clotting times compared to wildtype, whereas variants 138:160 and 136:160 were ˜3-fold less efficient than wildtype in generating a clot based upon their prolonged clotting times.

The degree of γ-carboxylation was investigated to determine if it has any rate alterations for macromolecular activity or clotting activity for the FVIIa variants (see, e.g., Neuenschwander and Morrissey “Roles of the membrane-interactive regions of Factor VIIa and tissue factor” J. Biol. Chem. 269: 8007-8013 (1994); Harvey et al. “Mutagenesis of the γ-carboxyglutamic acid domain of human factor VII to generate maximum enhancement of the membrane contact site” J. Biol. Chem. 278: 8363-8369 (2003)). However, there was essentially no difference in the degree of γ-carboxylation between the variants and wildtype as determined by mass spectrometry (Harvey et al. “Mutagenesis of the γ-carboxyglutamic acid domain of human factor VII to generate maximum enhancement of the membrane contact site” J. Biol. Chem. 278: 8363-8369 (2003)). See FIG. 6, Panels A and B.

Discussion: The mechanism underlying the zymogenicity of FVIIa and its dependence on cofactor TF for a full activity is only partially understood. See, e.g., Ruf and Dickinson “Allosteric regulation of the cofactor-dependent serine protease coagulation factor VIIa” Trends Cardiovasc. Med. 8:350-356 (1998); Eigenbrot “Structure, function and activation of coagulation FVII” Curr. Protein Peptide Sci. 3:287-299 (2002); Eigenbrot and Kirchhofer “New insight into how tissue factor allosterically regulates factor VIIa” Trends Cardiovasc. Med. 12:19-26 (2002); and, Petrovan and Ruf “Role of zymogenicity-determining residues of coagulation factor VII/VIIa in cofactor interaction and macromolecular substrate recognition” Biochemistry 41:9302-9309 (2002). Several engineered variants of FVIIa have been reported, revealing specific residues or regions on the protein that are important for its relationship with TF. To date, all reported mutations that improve enzymatic activity have been found either in the γ-carboxyglutamic acid (Gla) domain, resulting in FVII variants with significantly enhanced membrane binding properties and FX activation activity (Nelsestuen et al. “Elevated function of blood clotting factor VIIa variants that have enhanced affinity for membranes. Behavior in a diffusion-limited reaction” J. Biol. Chem. 276:39825-39831 (2001); and, Harvey et al. “Mutagenesis of the γ-carboxyglutamic acid domain of human factor VII to generate maximum enhancement of the membrane contact site” J. Biol. Chem. 278:8363-8369 (2003)), or one of three allosteric regions in the protease domain—the TF binding region of the FVIIa protease, the macromolecular substrate exosite and residues in the catalytic cleft. Alterations in one of these regions can have effect on the other areas, indicating that there is a direct yet complex set of interactions involved.

Previous strategies addressing FVIIa zymogenicity and engineering of rate enhancements for zymogen-like FVIIa have primarily involved substitution of residues from other intrinsically more active serine proteases. Met156 has been reported to be a determinant of FVIIa zymogenicity, since mutation to Gln, which is found at this position in FIX, resulted in 3- and 9-fold enhanced FVIIa amidolytic and proteolytic activity, respectively (see, Petrovan and Ruf “Residue Met156 contributes to the labile enzyme conformation of coagulation factor VIIa. J. Biol. Chem. 276:6616-6620 (2001)). Based upon a comparison of the free and TF-bound FVIIa structures (see, e.g., Pike et al. “Structure of human factor VIIa and its implications for the triggering of blood coagulation” Proc. Natl. Acad. Sci. USA 96:8925-8930 (1999), changing Leu163 to Val increased FVIIa activity 3- to 4-fold, presumably due to movement of the α-helix comprising residues 165-170 into an orientation more akin to that found in FXa or thrombin (Persson et al. “Substitution of valine for leucine 305 in factor VIIa increases the intrinsic enzymatic activity” J. Biol. Chem. 276:29195-29199 (2001). Additional residues targeted to stabilize this helix also increased activity, presumably by inducing a conformation similar to that found upon TF binding (Persson et al. “Augmented intrinsic activity of Factor VIIa by replacement of residues 305, 314, 337 and 374: Evidence of two unique mutational mechanisms of activity enhancement” Biochem. J. 379:497-503 (2004)). Mutations nearby the same α-helix also increased activity as found when the 170 loop in FVIIa was replaced by a shorter one from trypsin; replacement of the 99 loop with the corresponding sequence from trypsin also resulted in more active FVIIa variants and also broadened substrate specificity (Soejima et al. “Factor VIIa modified in the 170 loop shows enhanced catalytic activity but does not change the zymogen-like property” J. Biol. Chem. 276:17229-17235 (2001); and, Soejima et al. “The 99 and 170 loop-modified factor VIIa variants show enhanced catalytic activity without tissue factor” J. Biol. Chem. 277:49027-49035 (2002)). Mutation of Lys188 to Ala, designed to minimize repulsion of the positively charged N-terminus forming its salt bridge with Asp194 also resulted in FVIIa rate enhancement (Persson et al. “Rational design of coagulation factor VIIa variants with substantially increased intrinsic activity” Proc. Natl. Acad. Sci. U.S.A. 98:13583-13588 (2001)). The three-residue motif Val21, E154 and M156 in FVIIa was replaced by Glu, Arg and Lys respectively, found in thrombin and FIX, which also resulted in enhanced FVIIa activity (Persson et al. “Rational design of coagulation factor Vila variants with substantially increased intrinsic activity” Proc. Natl. Acad. Sci. U.S.A. 98:13583-13588 (2001b)). Mutations of these three ‘zymogenicity-determining’ residues have been studied more extensively, resulting in the conclusion that there is a complex set of interactions that stabilize the active conformation of FVIIa, but not zymogen FVII (Petrovan and Ruf “Role of zymogenicity-determining residues of coagulation factor VII/VIIa in cofactor interaction and macromolecular substrate recognition” Biochemistry 41:9302-9309 (2002)). Further studies have elucidated the molecular properties of these mutations (Persson and Olsen “Assignment of molecular properties of a superactive coagulation factor VIIa variant to individual amino acid changes” Eur. J. Biochem. 269:5950-5955 (2002)).

Here we have investigated a “mechanical rod” that can connect the TF binding site with segments of the activation domain and macromolecular substrate exosite. The elucidation of the zymogen structure of a shortened FVIIa, consisting of the second EGF-domain and the protease domain only, showed a three residues shift of β-strand B2 in the protease domain towards the C-terminus. This shift results in dramatic changes in the 170s loop and the preceding short α-helix that now would be impaired in contacting TF. As previously described, the H-bond interaction of β-strands B2 and A2 are almost identical to those seen in the enzyme structure and the suitability of side chain environments remained high based on the unique Leu-X-Val-Leu-X-Val sequence in B2 (Eigenbrot, C., et al., “The factor VII zymogen structure reveals reregistration of α-strands during activation” Structure 9:627-636 (2001)). In addition the H-bonds between Glu154 and Val21 and Cys22 prevent Ile16 from entering its hydrophobic pocket. All these features are indications for a stabilized zymogen-like form that could be accommodated even after activation cleavage in the absence of TF.

The enzymatic activity of FVIIa can be enhanced by engineering new a disulfide bond to restrict α-strand conformational changes. Orientations of the side chains as well as distances between the wildtype residues as seen in the crystal structures were considered to predict the mutations. Based on crystal structures of FVIIa and zymogen FVII this engineering design was considering a certain amount of rigidity of β-strand A2 that seems to remain the same conformation in both structures. A disulfide bond formation at the various double mutants would stabilize the strand shift, but could also restrain the region from further flexibility beyond the length of the disulfide bond. It is known that all the loops in the activation domain are highly flexible and undergo significant conformational changes between zymogen form and enzyme form. Without being limited to one theory, residues before and after these loops might not change their position significantly in the overall structure but their availability for a certain degree of flexibility may be important.

Several variants were designed and produced. Active protease-like conformations of FVIIa were engineered by placing cysteine residues into α-strands A2 and B2 to form a disulfide bond and a locked active enzyme conformation. Some of the substrates had enhanced amidolytic activity. The role of sTF was eliminated as a cofactor, thus achieving the goal of mimicking a TF•FVIIa-like conformational state with FVIIa itself. Engineered FVIIa can have advantageous properties as a therapeutic agent in certain clinical scenarios.

The specification is considered to be sufficient to enable one skilled in the art to practice the invention. It is understood that the examples and embodiments described herein are for illustrative purposes only. Indeed, various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

We claim:

1. A Factor VIIa (FVIIa) variant comprising an amino acid sequence derived from a mammalian FVIIa protein, wherein at least two amino acid residues are substituted with a cysteine amino acid.

2. The FVIIa variant of claim 1, wherein the at least two amino acid residues correspond to a human amino acid residue pair selected from the group consisting of S136 and V160, L137 and N159, V138 and V160, S139 and V157, F135 and N159, F135 and P161, V138 and L158, F135 and M156, and, V138 and L155.

3. The FVIIa variant of claim 1, wherein the FVIIa variant comprises an enhanced activity in the absence of tissue factor protein compared to a naturally occurring mammalian FVIIa protein.

4. The FVIIa variant of claim 1, wherein the at least two amino acid residues form a disulfide bond.

5. The FVIIa variant of claim 1, further comprising at least one additional amino acid substitution.

6. The FVIIa variant of claim 5, wherein the at least one additional amino acid substitution contributes to FVIIa variant activity.

7. The FVIIa variant of claim 5, wherein the at least one additional amino acid substitution corresponds to a change in the human amino acid residue selected from the group consisting of E17 (E154), V21 (V158), F135 (F278), S136 (S279), L137 (L280), V138 (V281), S139 (S282), E154 (E296), L155 (L297), M156 (M298), V157 (V299), L158 (L300), N159 (N301), V160 (V302), L163 (L305), M164 (M306), D167 (D309), S170b (S314), K188 (K337), and F225 (F374).

8. The FVIIa variant of claim 7, wherein the change in the human amino acid residue is selected from the group consisting of: V21D (V158D), V21E (V158E), V21N (V158N), E154V (E296V), E154I (E296I), E154R (E296R), M156Q (M298Q), M156K (M298K), L163V (L305V), M164D (M306D), D167S (D309S), S170bE (S314E), K188A (K337A), and F225Y (F374Y).

9. The FVIIa variant of claim 1, further comprising two or more additional amino acid substitutions.

10. The FVIIa variant of claim 1, wherein the mammalian FVIIa protein is a human FVIIa protein.

11. A Factor VIIa (FVIIa) variant comprising an amino acid sequence derived from a mammalian FVIIa protein, wherein at least two amino acid residues are substituted with an amino acid that locks A2-strand of FVIIa to B2-strand of FVIIa.

12. The FVIIa variant of claim 11, wherein the at least two amino acid residues corresponds to a human amino acid residue pair selected from the group consisting of: S136 and V160, L137 and N159, V138 and V160, S139 and V157, F135 and N159, F135 and P161, V138 and L158, F135 and M156, and, V138 and L155.

13. The FVIIa variant of claim 11, wherein the amino acid is a cysteine.

14. The FVIIa variant of claim 11, wherein the amino acid is an unnatural amino acid or modified amino acid.

15. A Factor VIIa (FVIIa) variant comprising an amino acid sequence derived from a mammalian FVIIa protein, wherein at least two amino acid residues are substituted with a cysteine amino acid, which correspond to a human amino acid residue pair S136 and V160.

16. A Factor VIIa (FVIIa) variant comprising an amino acid sequence derived from a mammalian FVIIa protein, wherein at least two amino acid residues are substituted with a cysteine amino acid, which correspond to a human amino acid residue pair L137 and N159.

17. A Factor VIIa (FVIIa) variant comprising an amino acid sequence derived from a mammalian FVIIa protein, wherein at least two amino acid residues are substituted with a cysteine amino acid, which correspond to a human amino acid residue pair V138 and V160.

18. A Factor VIIa (FVIIa) variant comprising an amino acid sequence derived from a mammalian FVIIa protein, wherein at least two amino acid residues are substituted with a cysteine amino acid, which correspond to a human amino acid residue pair S139 and V157.

19. A composition comprising a pharmaceutically acceptable excipient and the FVIIa variant of claim 1, 11, 15, 16, 17 or 18.

20. A method of altering procoagulation in a mammal comprising administering an effective amount of the composition of claim 19 to the mammal.

21. The method of claim 20, wherein the mammal is a human.

22. The method of claim 20, wherein the alteration is an induction of procoagulation.

23. An isolated DNA molecule encoding the FVIIa variant of claim 1, 11, 15, 16, 17 or 18.

24. The DNA molecule of claim 23, further comprising an expression control sequence operably linked to the DNA molecule.

25. An expression vector comprising the DNA molecule of claim 24, wherein the control sequence is recognized by a host cell with the introduced vector.

26. A host cell introduced with the vector of claim 25.

27. A method of producing a FVIIa variant, the method comprising: culturing the host cell of claim 26 under condition suitable for expression of the FVIIa variant, thereby producing the FVIIa variant.

28. The method of claim 27, further comprises recovering the FVIIa variant from the culture medium.
